SEQUENCE LISTING (139 sequences)

SEQ ID NO: 1 - RNA, 19 nt, unidentified
  source 1..19: mol_type = unassigned RNA
gggtgtacatacacccctt

SEQ ID NO: 2 - RNA, 19 nt, unidentified
  source 1..19: mol_type = unassigned RNA
gggtgtacatacacccctt

SEQ ID NO: 3 - RNA, 19 nt, unidentified
  source 1..19: mol_type = unassigned RNA
gcttccgtatatatggcat

SEQ ID NO: 4 - RNA, 19 nt, unidentified
  source 1..19: mol_type = unassigned RNA
ccctggctttcaacaccta

SEQ ID NO: 5 - RNA, 19 nt, unidentified
  source 1..19: mol_type = unassigned RNA
ggagtgacatccaggacaa

SEQ ID NO: 6 - RNA, 19 nt, unidentified
  source 1..19: mol_type = unassigned RNA
gggtctcactttccagcaa

SEQ ID NO: 7 - RNA, 19 nt, unidentified
  source 1..19: mol_type = unassigned RNA
ggtctcactttccagcaaa

SEQ ID NO: 8 - RNA, 19 nt, unidentified
  source 1..19: mol_type = unassigned RNA
gggaggtgctgaacagcat

SEQ ID NO: 9 - RNA, 19 nt, unidentified
  source 1..19: mol_type = unassigned RNA
cctgtttgctgtgtatgat

SEQ ID NO: 10 - RNA, 19 nt, unidentified
  source 1..19: mol_type = unassigned RNA
cccctggcctttgaggcaa

SEQ ID NO: 11 - RNA, 19 nt, unidentified
  source 1..19: mol_type = unassigned RNA
ccctggcctttgaggcaaa

SEQ ID NO: 12 - RNA, 19 nt, unidentified
  source 1..19: mol_type = unassigned RNA
cccccagtctcccaccttt

SEQ ID NO: 13 - RNA, 19 nt, unidentified
  source 1..19: mol_type = unassigned RNA
cctcccagtttgctgggtt

SEQ ID NO: 14 - RNA, 19 nt, unidentified
  source 1..19: mol_type = unassigned RNA
ccagtttgctgggtttatt

SEQ ID NO: 15 - RNA, 19 nt, unidentified
  source 1..19: mol_type = unassigned RNA
cgggactactgttccaaaa

SEQ ID NO: 16 - RNA, 19 nt, unidentified
  source 1..19: mol_type = unassigned RNA
cgaccagcttgtttgtgaa

SEQ ID NO: 17 - RNA, 19 nt, unidentified
  source 1..19: mol_type = unassigned RNA
gcattgccttcggtttgta

SEQ ID NO: 18 - RNA, 19 nt, unidentified
  source 1..19: mol_type = unassigned RNA
ggtttgtatttagtgtctt

SEQ ID NO: 19 - RNA, 19 nt, unidentified
  source 1..19: mol_type = unassigned RNA
gcttgtgatttttgaacaa

SEQ ID NO: 20 - RNA, 19 nt, unidentified
  source 1..19: mol_type = unassigned RNA
gcggaaccatagctggtta

SEQ ID NO: 21 - RNA, 19 nt, unidentified
  source 1..19: mol_type = unassigned RNA
tcatccacaatgagagtaa

SEQ ID NO: 22 - RNA, 21 nt, unidentified
  source 1..21: mol_type = unassigned RNA
gtcatccacaatgagagtaca

SEQ ID NO: 23 - DNA, 21 bp, synthetic construct
  source 1..21: mol_type = other DNA
  misc_feature 1..19: note = RNA
  misc_feature 20..21: note = DNA
aaggggtgtatgtacaccctt

SEQ ID NO: 24 - DNA, 21 bp, synthetic construct
  source 1..21: mol_type = other DNA
  misc_feature 1..19: note = RNA
  misc_feature 20..21: note = DNA
aagttttgcagcgactagctt

SEQ ID NO: 25 - DNA, 21 bp, synthetic construct
  source 1..21: mol_type = other DNA
  misc_feature 1..19: note = RNA
  misc_feature 20..21: note = DNA
atgccatatatacggaagctt

SEQ ID NO: 26 - DNA, 21 bp, synthetic construct
  source 1..21: mol_type = other DNA
  misc_feature 1..19: note = RNA
  misc_feature 20..21: note = DNA
taggtgttgaaagccagggtt

SEQ ID NO: 27 - DNA, 21 bp, synthetic construct
  source 1..21: mol_type = other DNA
  misc_feature 1..19: note = RNA
  misc_feature 20..21: note = DNA
ttgtcctggatgtcactcctt

SEQ ID NO: 28 - DNA, 21 bp, synthetic construct
  source 1..21: mol_type = other DNA
  misc_feature 1..19: note = RNA
  misc_feature 20..21: note = DNA
ttgctggaaagtgagaccctt

SEQ ID NO: 29 - DNA, 21 bp, synthetic construct
  source 1..21: mol_type = other DNA
  misc_feature 1..19: note = RNA
  misc_feature 20..21: note = DNA
tttgctggaaagtgagacctt

SEQ ID NO: 30 - DNA, 21 bp, synthetic construct
  source 1..21: mol_type = other DNA
  misc_feature 1..19: note = RNA
  misc_feature 20..21: note = DNA
atgctgttcagcacctccctt

SEQ ID NO: 31 - DNA, 21 bp, synthetic construct
  source 1..21: mol_type = other DNA
  misc_feature 1..19: note = RNA
  misc_feature 20..21: note = DNA
atcatacacagcaaacaggtt

SEQ ID NO: 32 - DNA, 21 bp, synthetic construct
  source 1..21: mol_type = other DNA
  misc_feature 1..19: note = RNA
  misc_feature 20..21: note = DNA
ttgcctcaaaggccaggggtt

SEQ ID NO: 33 - DNA, 21 bp, synthetic construct
  source 1..21: mol_type = other DNA
  misc_feature 1..19: note = RNA
  misc_feature 20..21: note = DNA
tttgcctcaaaggccagggtt

SEQ ID NO: 34 - DNA, 21 bp, synthetic construct
  source 1..21: mol_type = other DNA
  misc_feature 1..19: note = RNA
  misc_feature 20..21: note = DNA
aaaggtgggagactgggggtt

SEQ ID NO: 35 - DNA, 21 bp, synthetic construct
  source 1..21: mol_type = other DNA
  misc_feature 1..19: note = RNA
  misc_feature 20..21: note = DNA
aacccagcaaactgggaggtt

SEQ ID NO: 36 - DNA, 21 bp, synthetic construct
  source 1..21: mol_type = other DNA
  misc_feature 1..19: note = RNA
  misc_feature 20..21: note = DNA
aataaacccagcaaactggtt

SEQ ID NO: 37 - DNA, 21 bp, synthetic construct
  source 1..21: mol_type = other DNA
  misc_feature 1..19: note = RNA
  misc_feature 20..21: note = DNA
ttttggaacagtagtcccgtt

SEQ ID NO: 38 - DNA, 21 bp, synthetic construct
  source 1..21: mol_type = other DNA
  misc_feature 1..19: note = RNA
  misc_feature 20..21: note = DNA
ttcacaaacaagctggtcgtt

SEQ ID NO: 39 - DNA, 21 bp, synthetic construct
  source 1..21: mol_type = other DNA
  misc_feature 1..19: note = RNA
  misc_feature 20..21: note = DNA
tacaaaccgaaggcaatgctt

SEQ ID NO: 40 - DNA, 21 bp, synthetic construct
  source 1..21: mol_type = other DNA
  misc_feature 1..19: note = RNA
  misc_feature 20..21: note = DNA
aagacactaaatacaaacctt

SEQ ID NO: 41 - DNA, 21 bp, synthetic construct
  source 1..21: mol_type = other DNA
  misc_feature 1..19: note = RNA
  misc_feature 20..21: note = DNA
ttgttcaaaaatcacaagctt

SEQ ID NO: 42 - DNA, 21 bp, synthetic construct
  source 1..21: mol_type = other DNA
  misc_feature 1..19: note = RNA
  misc_feature 20..21: note = DNA
taaccagctatggttccgctt

SEQ ID NO: 43 - DNA, 21 bp, synthetic construct
  source 1..21: mol_type = other DNA
  misc_feature 1..19: note = RNA
  misc_feature 20..21: note = DNA
ttactctcattgtggatgatt

SEQ ID NO: 44 - RNA, 23 nt, synthetic construct
  source 1..23: mol_type = other RNA
tgtactctcattgtggatgacga

SEQ ID NO: 45 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gctagtcgctgcaaaactt

SEQ ID NO: 46 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ccctggctttcaacaccta

SEQ ID NO: 47 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggagtgacatccaggacaa

SEQ ID NO: 48 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gggtctcactttccagcaa

SEQ ID NO: 49 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 50 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 51 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 52 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 53 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 54 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcgctttccagcaaa

SEQ ID NO: 55 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 56 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gggaggtgctgaacagcat

SEQ ID NO: 57 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
cctgtttgctgtgtatgat

SEQ ID NO: 58 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
cccctggcctttgaggcaa

SEQ ID NO: 59 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ccctggcctttgaggcaaa

SEQ ID NO: 60 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
cccccagtctcccaccttt

SEQ ID NO: 61 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
cctcccagtttgctgggtt

SEQ ID NO: 62 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ccagtttgctgggtttatt

SEQ ID NO: 63 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
cgggactactgttccaaaa

SEQ ID NO: 64 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
cgaccagcttgtttgtgaa

SEQ ID NO: 65 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 66 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 67 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 68 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 69 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 70 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 71 - RNA, 19 nt, synthetic construct
  source 1..19: mol_type = other RNA
  modified_base 1: mod_base = OTHER; note = 2'-O-methyl modified nucleotide with phosphorothioate linkage attached to Peptide docking vehicle 3-N-Acetylgalactosamine
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gggtctcactttccagcaa

SEQ ID NO: 72 - RNA, 19 nt, synthetic construct
  source 1..19: mol_type = other RNA
  modified_base 1: mod_base = OTHER; note = 2'-O-methyl modified nucleotide with phosphorothioate linkage attached to Peptide docking vehicle 3-N-Acetylgalactosamine
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ggtctcactttccagcaaa

SEQ ID NO: 73 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to Peptide docking vehicle 3-N-Acetylgalactosamine
ggtctcactttccagcaaa

SEQ ID NO: 74 - RNA, 19 nt, synthetic construct
  source 1..19: mol_type = other RNA
  modified_base 1: mod_base = OTHER; note = 2'-O-methyl modified nucleotide with phosphorothioate linkage attached to Peptide docking vehicle 3-N-Acetylgalactosamine
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ccagtttgctgggtttatt

SEQ ID NO: 75 - RNA, 21 nt, synthetic construct
  source 1..21: mol_type = other RNA
  modified_base 1: mod_base = OTHER; note = 2'-O-methyl modified nucleotide with phosphorothioate linkage attached to Peptide docking vehicle 3-N-Acetylgalactosamine
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 76 - RNA, 21 nt, synthetic construct
  source 1..21: mol_type = other RNA
  modified_base 1: mod_base = OTHER; note = 2'-O-methyl modified nucleotide with phosphorothioate linkage attached to Peptide docking vehicle 3-N-Acetylgalactosamine
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
gtcatccacaatgagagtaca

SEQ ID NO: 77 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
aagttttgcagcgactagctt

SEQ ID NO: 78 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 18: mod_base = OTHER; note = nucleotide with phosphorothioate linkage
  modified_base 19: mod_base = OTHER; note = nucleotide with phosphorothioate linkage
  misc_feature 1..19: note = RNA
  misc_feature 20..21: note = DNA
taggtgttgaaagccagggtt

SEQ ID NO: 79 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttgtcctggatgtcactcctt

SEQ ID NO: 80 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttgctggaaagtgagaccctt

SEQ ID NO: 81 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 82 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 83 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-Fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 84 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-Fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 85 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..12: note = RNA
  misc_feature 13: note = DNA
  misc_feature 14..19: note = RNA
  misc_feature 20..21: note = DNA
tttgctggaaagtgagacctt

SEQ ID NO: 86 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..12: note = RNA
  misc_feature 13: note = DNA
  misc_feature 14..19: note = RNA
  misc_feature 20..21: note = DNA
tttgctggaaagtgagacctt

SEQ ID NO: 87 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..12: note = RNA
  misc_feature 13: note = DNA
  misc_feature 14..19: note = RNA
  misc_feature 20..21: note = DNA
tttgctggaaagtgagacctt

SEQ ID NO: 88 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
atgctgttcagcacctccctt

SEQ ID NO: 89 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
atcatacacagcaaacaggtt

SEQ ID NO: 90 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttgcctcaaaggccaggggtt

SEQ ID NO: 91 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgcctcaaaggccagggtt

SEQ ID NO: 92 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
aaaggtgggagactgggggtt

SEQ ID NO: 93 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 14: mod_base = OTHER; note = 2'-Fluoro modified nucleotide
  misc_feature 1..19: note = RNA
  misc_feature 20..21: note = DNA
aacccagcaaactgggaggtt

SEQ ID NO: 94 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
aataaacccagcaaactggtt

SEQ ID NO: 95 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttttggaacagtagtcccgtt

SEQ ID NO: 96 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttcacaaacaagctggtcgtt

SEQ ID NO: 97 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 98 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 99 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 100 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 101 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-Fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 102 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-Fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 103 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttgctggaaagtgagaccctt

SEQ ID NO: 104 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 105 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 106 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
aataaacccagcaaactggtt

SEQ ID NO: 107 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 108 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 109 - RNA, 19 nt, synthetic construct
  source 1..19: mol_type = other RNA
  modified_base 1: mod_base = OTHER; note = 5'-dibenzocyclooctyl-triethyleneglycol-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
gggtctcactttccagcaa

SEQ ID NO: 110 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttgctggaaagtgagaccctt

SEQ ID NO: 111 - RNA, 19 nt, synthetic construct
  source 1..19: mol_type = other RNA
  modified_base 1: mod_base = OTHER; note = 5'-dibenzocyclooctyl-triethyleneglycol-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ggtctcactttccagcaaa

SEQ ID NO: 112 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 113 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to amidated 1,2-dihexyldisulfane linker
ggtctcactttccagcaaa

SEQ ID NO: 114 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttgctggaaagtgagacctt

SEQ ID NO: 115 - RNA, 19 nt, synthetic construct
  source 1..19: mol_type = other RNA
  modified_base 1: mod_base = OTHER; note = 5'-dibenzocyclooctyl-triethyleneglycol-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ccagtttgctgggtttatt

SEQ ID NO: 116 - DNA, 21 bp, synthetic construct
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
aataaacccagcaaactggtt

SEQ ID NO: 117 - RNA, 21 nt, synthetic construct
  source 1..21: mol_type = other RNA
  modified_base 1: mod_base = OTHER; note = 5'-dibenzocyclooctyl-triethyleneglycol-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatccacaatgagagtaca

SEQ ID NO: 118 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 119 - RNA, 21 nt, synthetic construct
  source 1..21: mol_type = other RNA
  modified_base 1: mod_base = OTHER; note = 5'-dibenzocyclooctyl-triethyleneglycol-2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatccacaatgagagtaca

SEQ ID NO: 120 - RNA, 23 nt, synthetic construct
  source 1..23: mol_type = other RNA
  modified_base 1: mod_base = OTHER; note = Phosphorylated 2'-O-methyl modified nucleotide with phosphorothioate linkage
  modified_base 2: mod_base = OTHER; note = 2'-Fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
tgtactctcattgtggatgacga

SEQ ID NO: 121 - RNA, 19 nt, synthetic construct
  source 1..19: mol_type = other RNA
  modified_base 1: mod_base = OTHER; note = 5'-dibenzocyclooctyl-triethyleneglycol-2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
gggtctcactttccagcaa

SEQ ID NO: 122 - RNA, 19 nt, synthetic construct
  source 1..19: mol_type = other RNA
  modified_base 1: mod_base = OTHER; note = 5'-dibenzocyclooctyl-triethyleneglycol-2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ggtctcactttccagcaaa

SEQ ID NO: 123 - RNA, 19 nt, synthetic construct
  source 1..19: mol_type = other RNA
  modified_base 1: mod_base = OTHER; note = 5'-dibenzocyclooctyl-triethyleneglycol-2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ccagtttgctgggtttatt

SEQ ID NO: 124 - RNA, 21 nt, synthetic construct
  source 1..21: mol_type = other RNA
  modified_base 1: mod_base = OTHER; note = 5'-dibenzocyclooctyl-triethyleneglycol-2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatccacaatgagagtaca

SEQ ID NO: 125 - RNA, 21 nt, synthetic construct
  source 1..21: mol_type = other RNA
  modified_base 1: mod_base = OTHER; note = 5'-dibenzocyclooctyl-triethyleneglycol-2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatccacaatgagagtaca

SEQ ID NO: 126 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to aminated 5'-dibenzocyclooctyl via a 1,2-dihexyldisulfane linker
ggtctcactttccagcaaa

SEQ ID NO: 127 - RNA, 19 nt, synthetic construct
  source 1..19: mol_type = other RNA
  modified_base 1: mod_base = OTHER; note = 2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
gggtctcactttccagcaa

SEQ ID NO: 128 - RNA, 19 nt, synthetic construct
  source 1..19: mol_type = other RNA
  modified_base 1: mod_base = OTHER; note = 2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ggtctcactttccagcaaa

SEQ ID NO: 129 - RNA, 19 nt, synthetic construct
  source 1..19: mol_type = other RNA
  modified_base 1: mod_base = OTHER; note = 2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ccagtttgctgggtttatt

SEQ ID NO: 130 - RNA, 21 nt, synthetic construct
  source 1..21: mol_type = other RNA
  modified_base 1: mod_base = OTHER; note = 2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatccacaatgagagtaca

SEQ ID NO: 131 - RNA, 21 nt, synthetic construct
  source 1..21: mol_type = other RNA
  modified_base 1: mod_base = OTHER; note = 2'-O-methyl modified nucleotide attached to Peptide docking vehicle 3-N-Acetylgalactosamine
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
gtcatccacaatgagagtaca

SEQ ID NO: 132 - protein, 210 aa, synthetic construct
  VARIANT 1..200: note = This region may encompass 1-10 HnKm repeating units, wherein n is 1-10 and m is 1-10
  VARIANT 201..205: note = This region may encompass 1-5 residues
  VARIANT 206..210: note = This region may encompass 1-5 residues
  VARIANT 201..210: note = Any amino acid
HHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKXXXXXXXXXX

SEQ ID NO: 133 - protein, 17 aa, synthetic construct
KHHHKHHHKHHHKHHHK

SEQ ID NO: 134 - protein, 18 aa, synthetic construct
KHHHKHHHHKHHHKHHHK

SEQ ID NO: 135 - protein, 7 aa, synthetic construct
  SITE 1: note = Polyethylene glycol 3 malamide Lysine
KHHHCHH

SEQ ID NO: 136 - protein, 7 aa, synthetic construct
  SITE 1: note = Polyethylene glycol 3 malamide Lysine
  SITE 5: note = trivalent beta-N-Acetylgalactosamine polyethylene glycol maleimide modified residue
KHHHCHH

SEQ ID NO: 137 - protein, 7 aa, synthetic construct
  VARIANT 6: note = H or K
KHHHCXH

SEQ ID NO: 138 - protein, 7 aa, synthetic construct
  SITE 1: note = 1-azido-3,6,9,12-tetraoxapentadecan-15-oyl Lysine
  VARIANT 6: note = H or K
KHHHCXH

SEQ ID NO: 139 - protein, 7 aa, synthetic construct
  SITE 1: note = 1-azido-3,6,9,12-tetraoxapentadecan-15-oyl Lysine
  SITE 5: note = trivalent beta-N-Acetylgalactosamine polyethylene glycol maleimide modified residue
KHHHCHH

(19) United States
(12) Patent Application Publication
(10) Pub. No.: US 2024/0108741 A1
(43) Pub. Date: Apr. 4, 2024

(54) COMBINATION siRNA AND PEPTIDE DOCKING VEHICLE (PDoV) COMPOSITIONS AND METHODS OF THEIR USE FOR IMPROVED REGULATION AND FUNCTIONALITY

(71) Applicant: Sirnaomics, Inc., Germantown, MD (US)

(72) Inventors: Xiaoyong LU, Germantown, MD (US); David EVANS, Germantown, MD (US); Daniel MUTISYA, Germantown, MD (US)

(21) Appl. No.: 18/365,052

(22) Filed: Aug. 3, 2023

Related U.S. Application Data
(60) Provisional application No. 63/394,960, filed on Aug. 3, 2022.

(51) Int. Cl. A61K 47/64; A61P 9/12; C12N 15/113

(52) U.S. Cl. CPC A61K 47/6455; A61P 9/12; C12N 15/113; C12N 2310/14; C12N 2310/321; C12N 2310/322

(57) ABSTRACT

siRNA molecules targeting AGT mRNA that reduce or inhibit AGT production are provided. Pharmaceutical compositions containing such siRNA molecules also are provided, together with methods of their use for treating hypertension. Pharmaceutical compositions containing at least one oligonucleotide covalently linked to, and delivered to a target cell by, a peptide docking vehicle (PDoV) are capable of reducing or inhibiting the production of AGT and treating hypertension. The PDoV may contain a targeting ligand such as a GalNAc moiety conjugated to the PDoV which targets the complex to hepatocytes.

### RELATED APPLICATIONS

This application claims the benefit of and priority to US Provisional Patent Application No. 63/394,960, filed Aug. 3, 2022, which is incorporated herein by reference in its entirety.

### SEQUENCE LISTING

The instant application contains a Sequence Listing which has been submitted electronically in XML format and is hereby incorporated by reference in its entirety. Said XML document, created on Aug. 3, 2023, is named 4690_0077i_SL.xml and is 1,130,527 bytes in size.

### FIELD

Improved pharmaceutical compositions for siRNA delivery and efficacy are provided, together with methods of making and using the compositions.

### BACKGROUND

### Angiotensinogen (AGT) and the Regulation of Blood Pressure

The AGT gene encodes angiotensinogen, an upstream component of the renin-angiotensin-aldosterone system (RAAS), which regulates blood pressure and the balance of fluids and electrolytes in the body. Angiotensinogen is also known as SERPINA8 or ANHU, and is a member of the serpin family. (Gribouval et al., Nat. Genet. 2005, 37: 964-8).

Angiotensinogen is primarily secreted by the liver, and is a precursor to the angiotensin peptides, angiotensin I and angiotensin II. Angiotensinogen is converted by renin to angiotensin, which is then converted by angiotensin converting enzyme (ACE) to angiotensin II (Gribouval et al., Hum. Mutat. 2012, 33: 316-26). Angiotensin II causes blood vessels to narrow, which results in increased blood pressure. Angiotensin II also stimulates production of the hormone aldosterone, which triggers the absorption of salt and water by the kidneys. The increased amount of fluid in the body also increases blood pressure. In addition, angiotensin II may play a more direct role in kidney development, perhaps by affecting growth factors involved in the development of kidney structures. (Gubler et al., Kidney Int. 2010, 77: 400-6). Over-stimulation or activity of the RAAS pathway can cause high blood pressure. Chronic high blood pressure is known as hypertension, and this condition means the heart must work harder to circulate blood through the blood vessels.

### Hypertension and Treatment

Approximately 26 percent of the adult population worldwide suffers from chronic hypertension. If untreated, hypertension can lead to stroke, or heart or kidney failure, and death. Several antihypertension drugs currently on the market primarily target the gene products in RAAS. Successful treatment of hypertension usually requires administration of multiple antihypertensive drugs, but treatment becomes more complicated with every drug added to the treatment combination (Kearney et al., J Manag Care Pharm. 2007, 13(Suppl B), 9-20).

Pharmaceutical agents against major RAAS pathway components have been successful in lowering blood pressure and improving outcomes in patients with cardiovascular diseases.

The treatment of hypertension is complicated, however, by the upregulation of counterbalancing mechanisms, for example, the rise in renin during blockade of the RAAS.

As a consequence, Angiotensin II levels are often restored to their original (elevated), pretreatment levels during the chronic treatment phase (van den Meiracker et al., Hypertension. 1995, 25: 22-29; Sealey et al., Am J Hypertens. 2007;20:587-597). Thus, the blockade of the RAAS at this (downstream) level may ultimately extend or worsen hypertension.

AGT, the most upstream component of the RAAS pathway, has until now not been targeted due to the difficulty of modulating its expression through current pharmacological interventions. Nevertheless, a reduction in plasma AGT levels would be expected to dampen activity of the RAAS pathway, as other clinical agents do and, therefore, lower blood pressure. Thus, modulation of RAAS upstream at the level of AGT using an alternative approach offers a potentially useful and superior treatment for hypertension, one that is potentially free of the complications seen with current pharmaceutical approaches.

### RNA Interference (RNAi)

RNA interference (RNAi) is a post-transcriptional gene silencing mechanism that uses small double-stranded RNA molecules to direct gene silencing in a homology-based manner. Small interfering RNAs (siRNAs) recruit an RNA-induced silencing complex to the target mRNA, which then undergoes site-specific cleavage and degradation (Estrellita Uijl et al., Hypertension. 2019, 73:1249-1257). Ongoing efforts to develop siRNA drug therapeutics have now yielded hepatocyte-directed, N-acetylgalactosamine (GalNAc)-conjugated molecules with long-term, single-dose efficacy. Short-interfering RNA (siRNA)-induced RNAi regulation shows great potential to treat a wide variety of human diseases from cancer to other traditional undruggable disease. Onpattro (patisiran) infusion is approved for the treatment of peripheral nerve disease (polyneuropathy) caused by hereditary transthyretin-mediated amyloidosis (hATTR) in adult patients. This is the first FDA approved siRNA based drug treatment for patients with polyneuropathy caused by hATTR, a rare, debilitating and often fatal genetic disease.

In addition, it was discovered that tri-antennary N-acetylgalactosamine (GalNAc) could mediate highly efficient targeted delivery of siRNAs to hepatocytes via binding to the asialoglycoprotein receptor. siRNA delivery to the liver can be achieved using ASGPR-targeted GalNAc-siRNA conjugates since hepatocytes express millions of copies of ASGPR on the cell surface, which cycle at a rapid rate of every 10-15 min. One approved RNAi therapeutic employing GalNAc delivery is Givlaari (givosiran) for acute hepatic porphyria (AHP), a rare inherited genetic disease. Givosiran binds to and suppresses the translation of delta aminolevulinic acid synthase 1 (ALAS1) mRNA, thereby reducing the neurotoxic intermediates in this disease. Oxlumo™ (lumasiran) is approved in the EU for the Treatment of Primary Hyperoxaluria Type 1 in all age groups. Another approved siRNA drug is Inclisiran, that targets PCSK9 for the treatment of hypercholesterolemia. In 2022, Amvuttra™ (vutrisiran), an RNAi therapeutic for the Treatment of the Polyneuropathy of Hereditary Transthyretin-Mediated Amyloidosis, is another approved drug that employs GalNAc delivery. There are currently many ongoing clinical trials for RNAi-related drugs.

Currently, there is no clinically marketed drug based AGT siRNA-targeted approach for treatment of hypertension. Subcutaneously delivered AGT targeted antisense oligonucleotides (ASO) and intravenous injection of nanoparticles containing small interfering RNAs (siRNAs) targeting AGT effectively lower blood pressure in hypertensive animal and clinical studies. There is still an unmet need to develop alternative treatments to inhibit the RAAS pathway and treat hypertension.

### SUMMARY OF THE INVENTION

What is provided is a conjugate containing a peptide covalently coupled to both an siRNA duplex and an N-acetyl-galactosamine (GalNAc) moiety, where the siRNA duplex consists essentially of a duplex containing 14-25 duplex residues of an siRNA selected from the group consisting of (a) AGT01-AGT22, where the duplex is modified or unmodified and (b) M02, M04-M16, M21, M21a, APG3M06, APG3M07, APG3M07a, and APG3M14. The siRNA duplex may consist essentially of a duplex containing 14-25 duplex residues of an siRNA selected from the group consisting of AGT01-AGT20, where at least one residue of the duplex is modified. The modification may be, for example, a 2′-O-methyl or 2′-F modification.

In these conjugates the siRNA duplex may be, for example, M06, M07, M07a or M14. The modified oligonucleotide may contain at least one modification selected from a modified GalNAc moiety and a peptide linkage. The siRNA duplex may contain a sense strand that is at least 90% complementary to a nucleobase antisense sequence selected from the group consisting of the antisense sequences of AGT01-AGT22. The siRNA duplex may contain a dTdT overhang at the 3′ end of one or both of the strands and/or may contain a phosphate modification at the 5′ end of the antisense strand. In certain conjugates the siRNA may contain at least two phosphorothioates modifications at the end of at least one strand.

In these conjugates the siRNA molecule may be via the 5′ end of one strand, such as the sense strand, or may be coupled via the 3′ end of one strand.

The conjugate may contain a trivalent GalNAc motif having the structure:

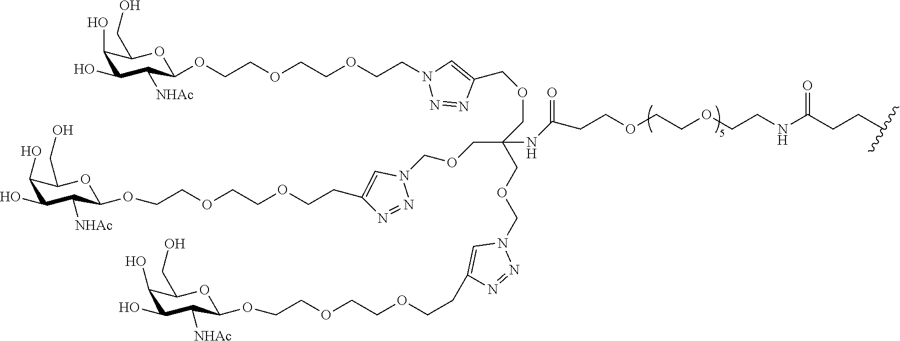

The peptide of the conjugate may contain the structure disclosed as SEQ ID NO: 135:

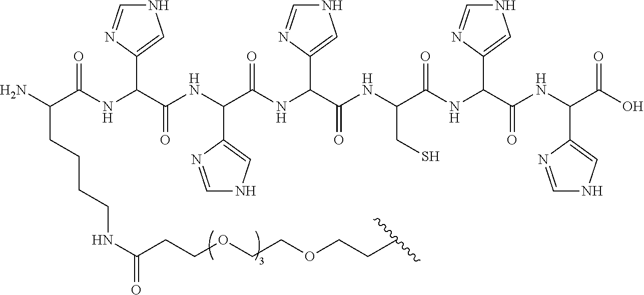

In some conjugates, the peptide may be coupled via a peg linker covalently linked to the side chain of a lysine residue. The peg linker may be, for example, —(CH₂CH₂)₃—OCH₂CH₂—N—.

The peptide moiety of the conjugate may contain a cysteine residue.

A specific example of a conjugate has the structure disclosed as SEQ ID NO: 136:

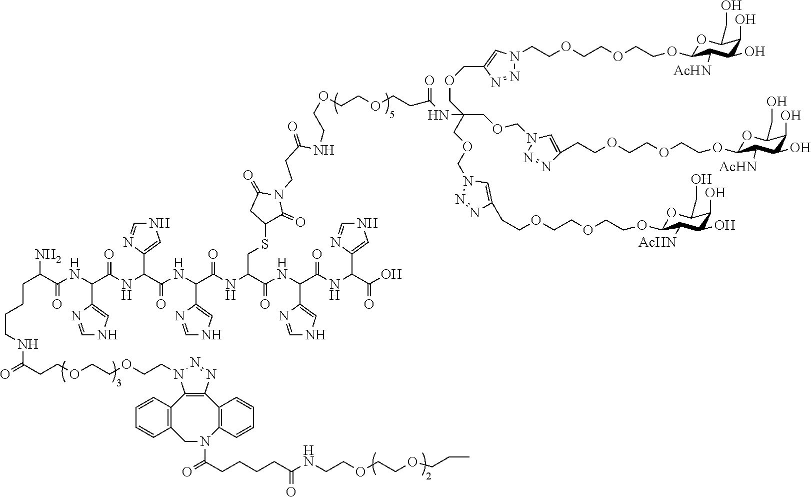

Also provided are pharmaceutical compositions containing the conjugate as described above, together with a pharmaceutically acceptable carrier or diluent. The pharmaceutical composition may contain an HKP copolymer and may be formulated into nanoparticles.

Further provided are methods of treating hypertension in a subject by administering to the subject an effective amount of a conjugate or pharmaceutical composition as described above. The subject may be a primate, for example a human.

### BRIEF DESCRIPTION OF THE DRAWINGS

FIG. 1. siRNA sequence design (unmodified, SEQ ID Nos. 1-44).

FIGS. 2A-2E. siRNA sequence design and chemical modification: (FIG. 2A): Table 2, modified siRNA sequences (SEQ ID Nos 45-108); (FIG. 2B): Table 3, AS and SS sequences with additional modifications resulting from synthesis as described in Example 8 (SEQ ID Nos. 109-120); (FIG. 2C): Table 4, sequences synthesized with 5′ GalNAc-PDoV as described in Example 9 (SEQ ID Nos.121-125); (FIG. 2D): Table 5, sequence synthesized with DBCO-NH-C6 as described in Example 10 (SEQ ID No. 126); (FIG. 2E): Table 6: AGT duplexes; sense strands from Table 2A, but not showing some 2′OMe in the sequences themselves; see legend to Table 6 below. Table 2A shows SEQ ID Nos 127, 128, 129, 130 and 131 in Table 6, as SEQ ID Nos. 71, 72, 74, 75, and 76, respectively, with all modifications in the sequences themselves.

FIG. 3. Three-point screening of AGT siRNA in HepG2 cell line by lipofectamine transfection. 1=50 nM, 2=5 nM, 3=0.5 nM. Knockdown efficacy study of unmodified siRNA (AGT01, AGT02, AGT03, AGT04, AGT05, AGT06, AGT07, AGT08, AGT09, AGT10, AGT11, AGT12, AGT13, AGT14, AGT15, AGT16, AGT17, AGT18, AGT19, AGT20, AGT21 and AGT22 (ag002), SEQ ID Nos. 1-44). HepG2: 2×10⁵ cells per well in 12-well plate in transfection time for 48 hr. siRNA concentrations in transfection: 50 nM, 5 nM, 0.5 nM.

FIG. 4. Twelve-point titration of AGT siRNA in HepG2 cell line by lipofectamine transfection. Knockdown efficacy study of AGT siRNA (AGT05, AGT06, AGT08, AGT12, AGT13, and AGT22 (ag002), SEQ ID Nos. 5, 27, 6, 28, 8, 30, 12, 34, 13, 35, 22 and 44, respectively) by Serial dilution. In vitro knockdown experiment was performed in HepG2 cell line in 2 x 10 5 cells per well in 12-well plate. siRNA final concentrations 10× dilution were 1=1 μM, 2=0.2 μM, 3=0.04 μM, 4=0.008 μM, 5=0.0016 μM, 6=0.00032 μM, 7=0.000064 μM. Cells were incubated for 48 hr in transfection. RT PCR was performed by cDNA synthesis (per reaction): Maxima First strand cDNA synthesis Kit (Cat #1672).

FIG. 5. IC50 fitting for the 12 point titration of AGT siRNA in HepG2 cell line by lipofectamine transfection. IC50 fitting calculation of knockdown efficacy study of AGT siRNA (AGT05, AGT06, AGT08, AGT12, AGT13, and AGT22 (ag002), SEQ ID Nos. 5, 27, 6, 28, 8, 30, 12, 34, 13, 35, 22 and 44, respectively) by Serial dilution. In vitro knockdown experiment was performed in HepG2 cell line in 2×10⁵ cells per well in 12-well plate. siRNA final concentrations 10× dilution were 1=1 μM, 2=0.2 μM, 3=0.04 μM, 4=0.008 μM, 5=0.0016 μM, 6=0.00032 μM, 7=0.000064 μM. Cells were incubated for 48 hr in transfection.

FIG. 6. Twelve-point itration of AGT siRNA in HepG2 cell line by lipofectamine transfection. Knockdown efficacy study of AGT siRNA (AGT02, AGT04, AGT07, AGT14, AGT15, AGT16 and AGT22 (ag002), SEQ ID Nos. 2, 24, 4, 26, 7, 29, 14, 36, 15, 37, 16, 38, 22 and 44, respectively) by Serial dilution. In vitro knockdown experiment was performed in HepG2 cell line in 2×10⁵ cells per well in 12-well plate. siRNA final concentrations 10× dilution were 1=1 μM, 2=0.2 μM, 3=0.04 μM, 4=0.008 μM, 5=0.0016 μM, 6=0.00032 μM, 7=0.000064 μM. Cells were incubated for 48 hr in transfection. RT PCR was performed by cDNA synthesis (per reaction): Maxima First strand cDNA synthesis Kit (Cat #1672).

FIG. 7. IC50 fitting for the 12 point titration of AGT siRNA in HepG2 cell line by lipofectamine transfection. IC50 fitting calculation of knockdown efficacy study of AGT siRNA (AGT02, AGT04, AGT07, AGT14, AGT15, AGT16 and AGT22 (ag002), SEQ ID Nos. 2, 24, 4, 26, 7, 29, 14, 36, 15, 37, 16, 38, 22 and 44, respectively)by Serial dilution. In vitro knockdown experiment was performed in HepG2 cell line in 2×10⁵ cells per well in 12-well plate. siRNA final concentrations 10× dilution were 1=1 μM, 2=0.2 μM, 3=0.04 μM, 4=0.008 μM, 5=0.0016 μM, 6=0.00032 μM, 7=0.000064 μM. Cells were incubated for 48 hr in transfection.

FIG. 8. Twelve-point titration of AGT modified siRNAs in HepG2 Cells for 48 hr, Multiplex PCR. Knockdown efficacy study of AGT siRNA (M07, M12, M13, M14, M15 and M21, SEQ ID Nos. 49, 81, 60, 92, 61, 93, 62, 94, 63, 95, 65 and 97, respectively, in Table 2A) by Serial dilution. In vitro knockdown experiment was performed in HepG2 cell line in 2×10⁵ cells per well in 12-well plate. siRNA final concentrations 10× dilution were 1=1 μM, 2=0.2 μM, 3=0.04 μM, 4=0.008 μM, 5=0.0016 μM, 6=0.00032 μM, 7=0.000064 μM, 8=0.0000128 μM, 9=0.00000256 μM, 10=0.000000512 μMm, 11=0.0000001024 μM, and 12=0.00000002048 μM. Cells were incubated for 48 hr in transfection. RT PCR was performed by cDNA synthesis (per reaction): Maxima First strand cDNA synthesis Kit (Cat #1672).

FIG. 9. Screening of AGT-PDoV3 conjugated siRNA in HepG2 cells with addition of lipofectamine. 1=50 nM, 2=5 nM, 3=0.5 nM. Cell transfection: 48 hours. Samples include WT06 SEQ ID Nos. 6, 28), WT07 (SEQ ID Nos. 7, 29), WT14 (SEQ ID No. 14, 36), M06 (SEQ ID Nos. 48, 80), M07 (SEQ ID Nos. 49, 81); M09 (SEQ ID Nos. 57, 89); M14 (SEQ ID Nos. 62, 94), M21 (SEQ ID Nos. 65, 98), M21a (SEQ ID Nos. 66, 98), APG3M06 (SEQ ID Nos. 71, 103); APG3M07 (SEQ ID Nos. 72, 104); APG3M07a (SEQ ID Nos. 73, 105); APG3M14 (SEQ ID Nos. 74, 106); APG3M21(SEQ ID Nos. 75, 107); and APG3M21a (SEQ ID Nos. 76, 108).

FIG. 10. Screening of AGT-PDoV3 conjugated siRNA in human primary hepatocytes. Knockdown efficacy study of AGT siRNA (APG3M06 (SEQ ID Nos. 71, 103), APG3M07 (SEQ ID Nos. 72, 104), APG3M07a (SEQ ID Nos. 73, 105), APG3M14 (SEQ ID Nos. 74, 106), APG3M21(SEQ ID Nos. 75, 107), and APG3M21a (SEQ ID Nos. 76, 108) by Serial dilution. In vitro knockdown experiment was performed in human primary hepatocytes cell line in 2×10⁵ cells per well in 12-well plate. siRNA final concentrations 10× dilution were 1=1 μM, 2=0.2 μM, 3=0.04 μM, 4=0.008 μM, 5=0.0016 μM, 6=0.00032 μM, 7=0.000064 μM. Cells were incubated for 48 hr in transfection. RT PCR was performed by cDNA synthesis (per reaction): Maxima First strand cDNA synthesis Kit (Cat #1672).

FIG. 11. Scheme of synthesis of the GalNAc-PDoV3-ligand. Shown are the steps of preparation of GalNAc-PDoV3-ligand which is used to conjugate with the siRNA. Figure discloses SEQ ID NOS 135 and 139, respectively, in order of appearance.

FIG. 12. Scheme of synthesis of 5′-GalNAc-PDoV3-sense strand oligonucleotide. Conjugates APG3M06 (SEQ ID Nos. 71, 103), APG3M07 (SEQ ID Nos. 72, 104), APG3M07a (SEQ ID Nos. 73, 105), APG3M14 (SEQ ID Nos. 74, 106), APG3M21(SEQ ID Nos. 75, 107), and APG3M21a (SEQ ID Nos. 76, 108) are prepared by the similar method. Figure discloses SEQ ID NOS 139 and 136, respectively, in order of appearance.

FIG. 13. Scheme of synthesis of 3′-GalNAc-PDoV3-sense strand oligonucleotide. The conjugate, APG3M07a (SEQ ID Nos. 73 and 105), is prepared by this method. Figure discloses SEQ ID NOS 139 and 136, respectively, in order of appearance.

FIG. 14. Relative signal from screening AGT-PDoV3 conjugated siRNA (WT09, SEQ ID Nos. 9, 31; WT14, SEQ ID Nos. 14, 36; WT15, SEQ ID Nos. 15, 37, M09, SEQ ID Nos. 57, 89; M14 SEQ ID Nos. 62, 94; M15, SEQ ID Nos. 63, 95; and M21a, SEQ ID Nos. 66, 98) in HepG2 cell line with addition of lipofectamine. 1=50 nM, 2=5 nM, 3=0.5 nM.

FIG. 15. Twelve-point titration of AGT siRNA (M02, SEQ ID Nos. 45, 77; M05, SEQ ID Nos. 47, 79; M06, SEQ ID Nos. 48, 80; M07, SEQ ID Nos. 49, 81, and AGT22 (ag002), SEQ ID Nos. 22, 44) in HepG2 Cells for 48 hr, Multiplex PCR, 1=1 μM, 2=0.2 μM, 3=0.04 μM, 4=0.008 μM, 5=0.0016 μM, 6=0.00032 μM, 7=0.000064 μM, 8=0.0000128 μM, 9=0.00000256 μM, 10=0.000000512 μMm, 11=0.0000001024 μM, and 12=0.00000002048 μM.

FIG. 16. IC50 fitting for the 12 point titration of AGT siRNA (M02, SEQ ID Nos. 45, 77; M05, SEQ ID Nos. 47, 79; M06, SEQ ID Nos. 48, 80; M07, SEQ ID Nos. 49, 81, and AGT22 (ag002), SEQ ID Nos. 22, 44) in HepG2 cell line by lipofectamine transfection for identified siRNAs.

FIG. 17. Twelve-point titration of AGT siRNA (M07, SEQ ID Nos. 49, 81; M07c1, SEQ ID Nos. 50, 82; M07d1, SEQ ID Nos. 51, 83; M07f1, SEQ ID Nos. 52, 84; M07m, SEQ ID Nos. 53, 85; M07m1, SEQ ID Nos. 54, 86; M07m2, SEQ ID Nos. 55, 87) in HepG2 Cells for 48 hr, Multiplex PCR, 1=200 μM, 2=0.04 μM, 3=0.008 μM, 4=0.0016 μM, 5=0.00032 μM, 6=0.000064 μM, 8=0.0000128 μM, 9=0.00000256 μM, 10=0.000000512 μMm, 11=0.0000001024 μM, and 12=0.00000002048 μM.

FIG. 18. IC50 fitting for the 12 point titration of AGT siRNA (M07, SEQ ID Nos. 49, 81; M07c1, SEQ ID Nos. 50, 82; M07d1, SEQ ID Nos. 51, 83; M07f1, SEQ ID Nos. 52, 84; M07m, SEQ ID Nos. 53, 85; M07m1, SEQ ID Nos. 54, 86; M07m2, SEQ ID Nos. 55, 87) in HepG2 cell line by lipofectamine transfection for siRNAs M07-c1, M07-d1, M07-f1, M07m, M07-m1, M07-m2 and M07.

### DETAILED DESCRIPTION

siRNA molecules targeting the AGT mRNA that reduce or inhibit AGT production are provided. Pharmaceutical compositions containing such siRNA molecules also are provided, together with methods of their use for treating hypertension. Pharmaceutical compositions containing at least one oligonucleotide covalently linked to, and delivered to a target cell by, a peptide docking vehicle (PDoV) are capable of reducing or inhibiting the production of AGT and treating hypertension. The PDoV may contain a targeting ligand directed to a target cell, for example it may contain a GalNAc moiety conjugated to the PDoV which targets the complex to hepatocytes.

### Definitions:

As used herein, “oligonucleotide” refers to a chemically modified or unmodified nucleic acid molecule (RNA or DNA) having a length of less than 100 nucleotides (for examples less than 50 nucleotides). It can be siRNA, microRNA, antimicroRNA, microRNA mimics, dsRNA, ssRNA, aptamer, triplex forming oligonucleotides, or aptamers. Advantageously the oligonucleotide is an RNAi agent.

As used herein, an “siRNA molecule” is a duplex oligonucleotide, that is a short, double-stranded polynucleotide, that interferes with the expression of a gene in a cell, after the molecule is introduced into the cell. For example, it targets and binds to a complementary nucleotide sequence in a single stranded target RNA molecule. siRNA molecules are chemically synthesized or otherwise constructed by techniques known to those skilled in the art. Such techniques are described, for example, in U.S. Pat. Nos. 5,898,031, 6,107,094, 6,506,559, 7,056,704 and in European Pat. Nos. 1214945 and 1230375. By convention in the field, when an siRNA molecule is identified by a particular nucleotide sequence, the sequence refers to the sense strand of the duplex molecule. One or more, or all, of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g., sugar molecules), amino acids, peptides, cholesterol, and other large molecules for conjugation onto the siRNA molecule.

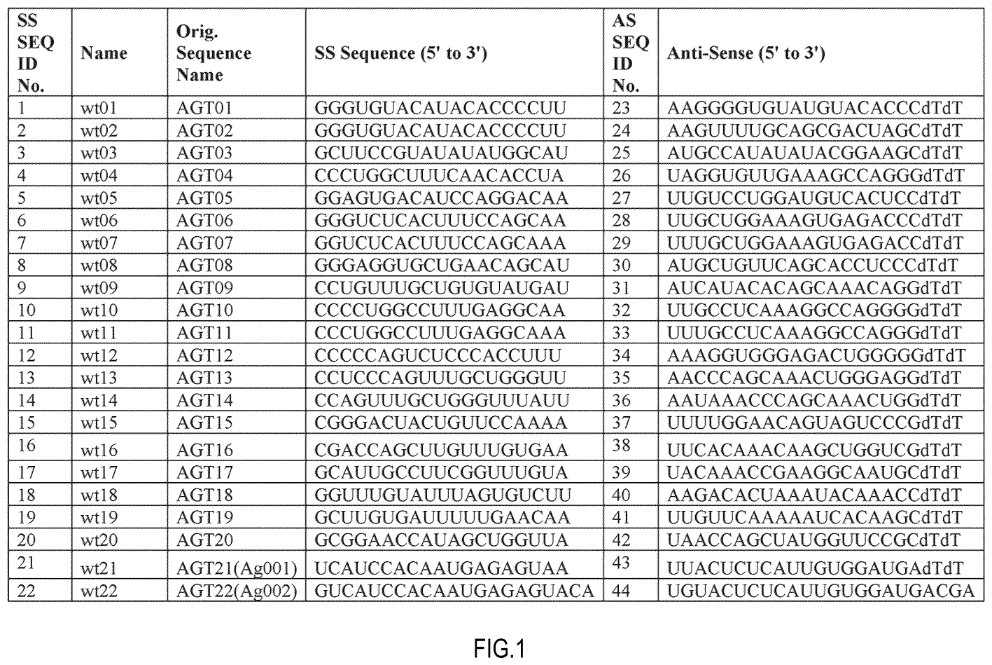

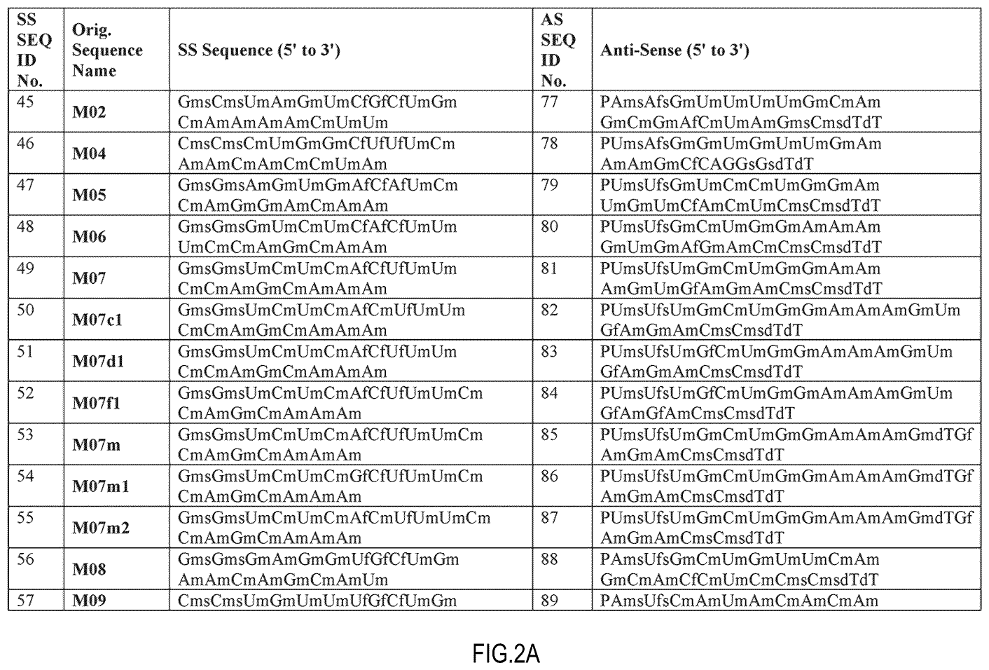

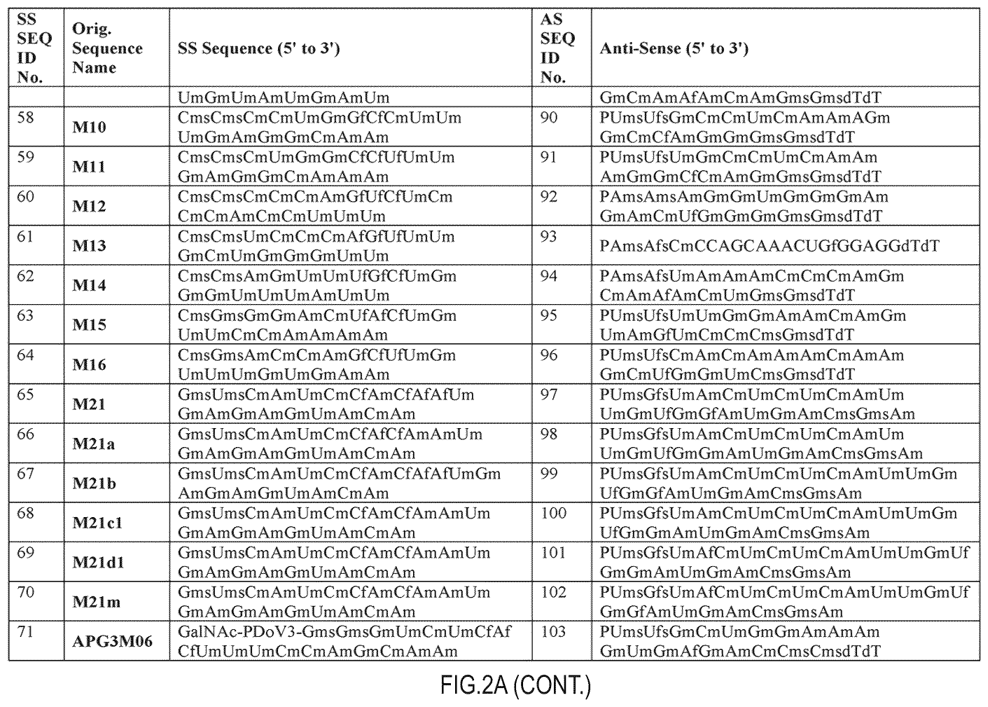

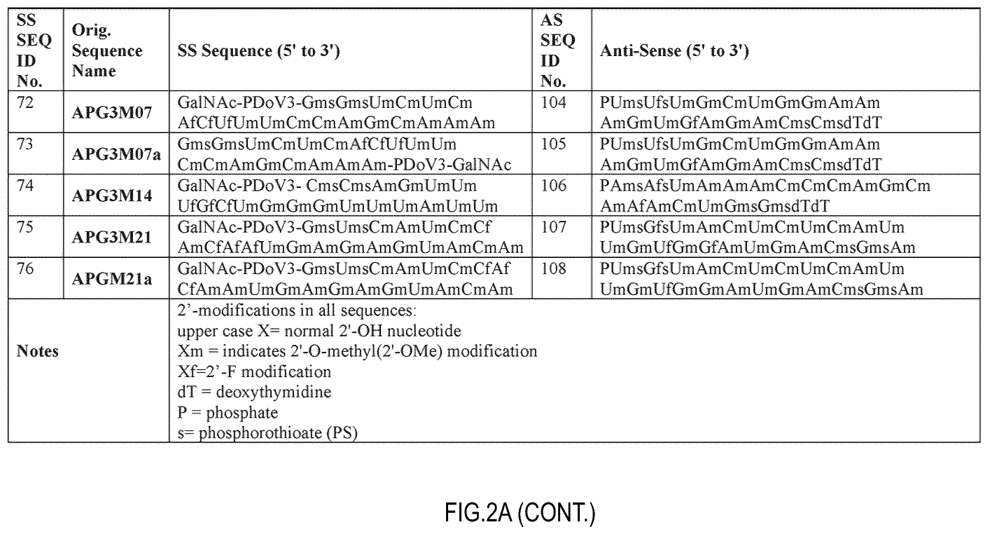

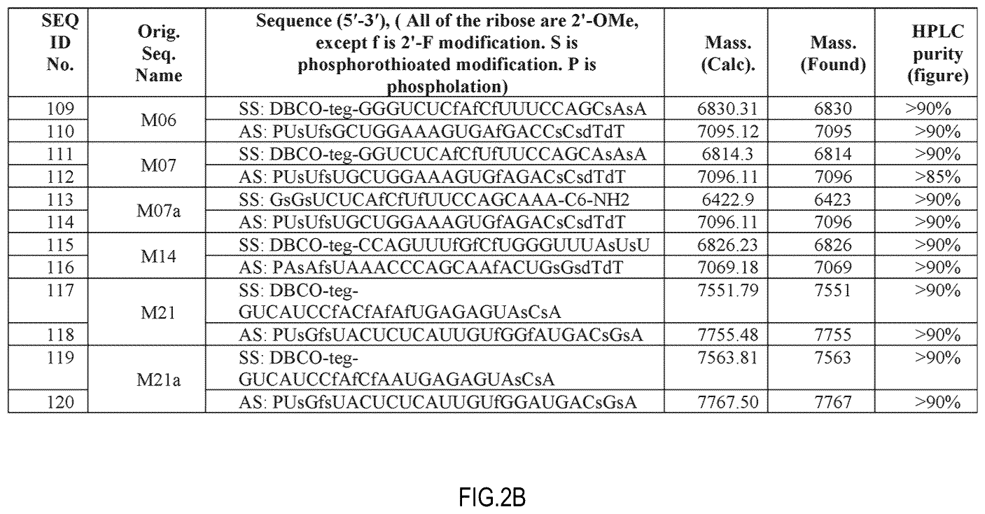

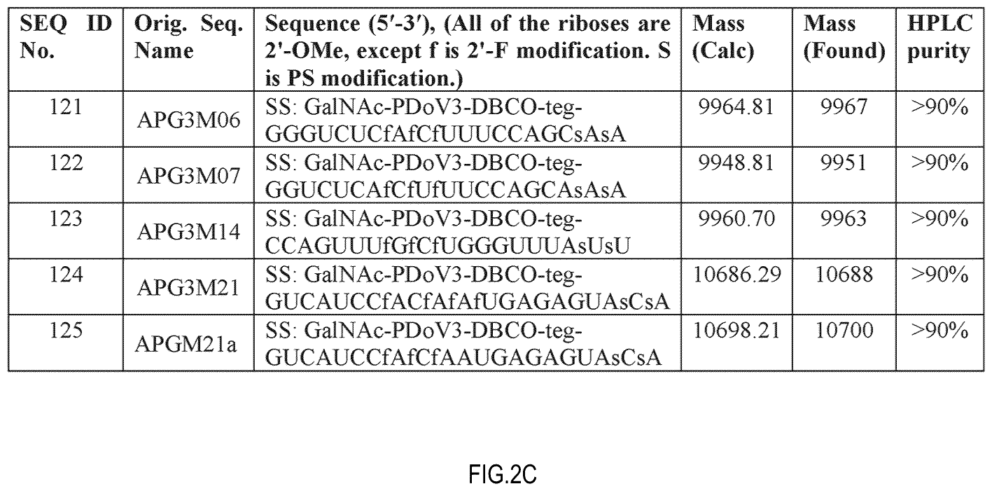

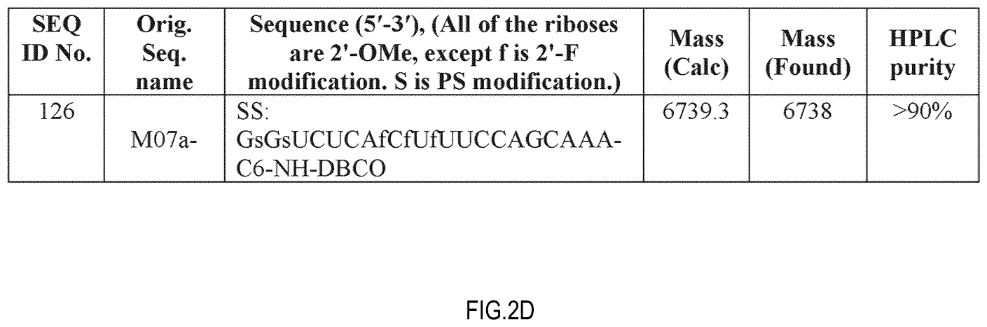

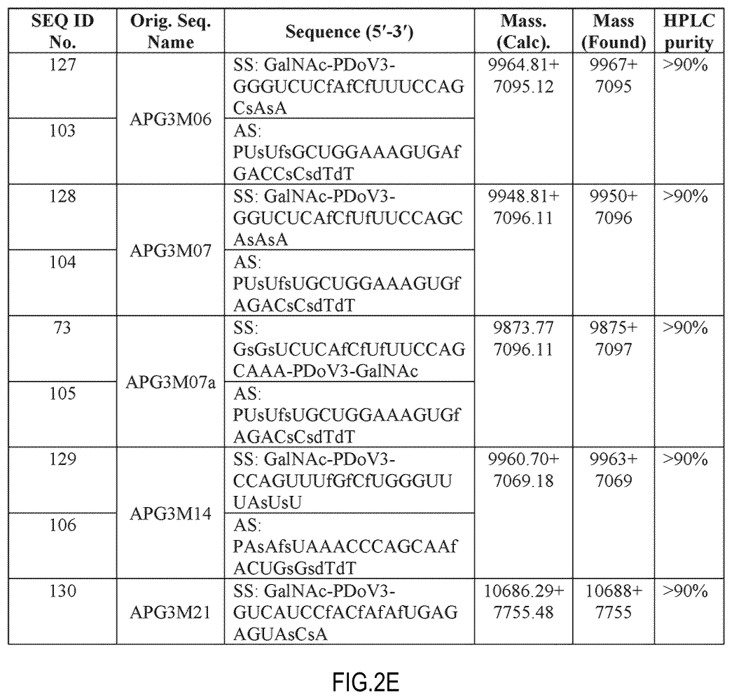

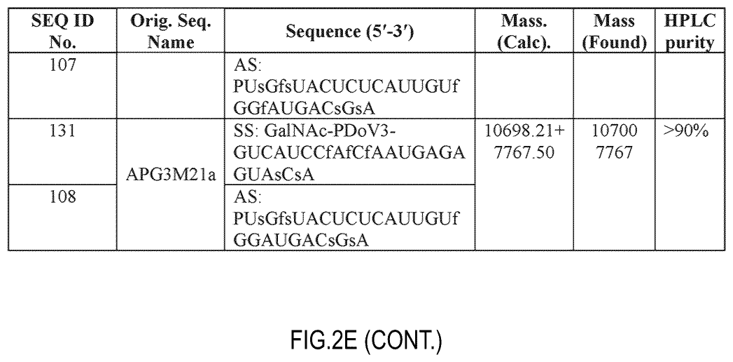

RNAi Agents : RNAi molecules are double stranded compounds. The double stranded siRNA can be anti AGT, and can be unmodified or chemically modified at the 2′ position with, for example, 2′-OCH3, 2′-F, or 2′-O-MOE, or at the 5′ position with -P(O)2=S. Other chemical modifications are known in the art and can include, for example, pegylation or lipid functionalization to improve the overall stability and bioavailability of the RNAi. In specific embodiments, the double stranded siRNA may be duplexes consisting of 24, 23, 22, 21, 20, 19, 18, 17 or 16 contiguous base pairs of any one or more of the duplexes in FIG. 1 and FIG. 2.

In other embodiments, the siRNA molecule contains a duplex of two complimentary, single-stranded oligonucleotides that have the same length and where each oligonucleotide has a length of 10-29 bases or 19-27 bases. The duplexes may be blunt-ended or may have 1 or 2 base overhangs at the 3′ end of one or both strands. Advantageously the overhangs are dTdT.

A modified GalNAc moiety: The targeting ligand moiety may be, for example, N-acetyl-galactosamine (GalNAc). The targeting ligands are coupled to the peptide via a covalent bond. The targeting ligand may contain 2 or 3 GalNAc ligands. The targeting ligands disclosed here advantageously have the following structure (shown below as the maleimide-containing reagent used to conjugate the ligand to an siRNA molecule:

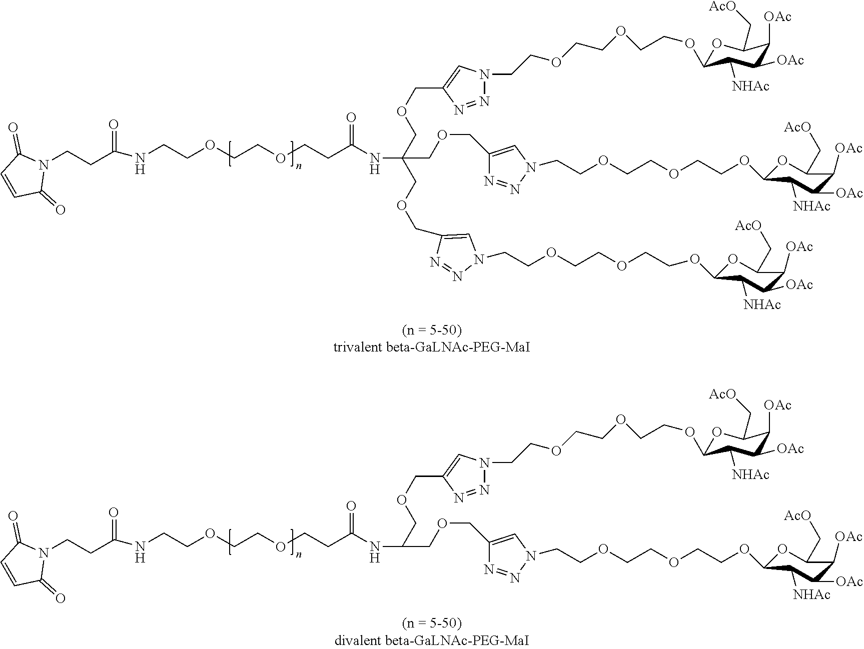

A peptide docking linkage: “Peptide Docking Vehicle” (PDoV) refers to a synthetic peptide of defined sequence that contains multiple conjugation sites to allow conjugation with one or more targeting ligands and with one or more oligonucleotides. It contains functional groups, such as a hydrophobic chain or a pH sensitive residue, which facilitate the release of the oligonucleotide payload entrapped inside of the endosome of a cell after delivery of the conjugated PDoV to the cell.

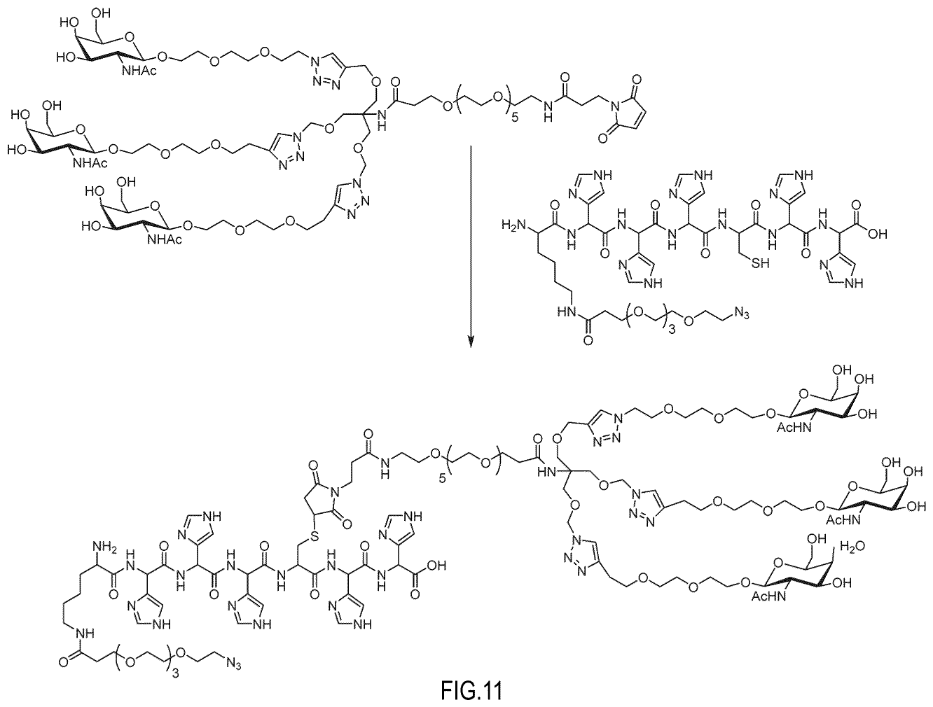

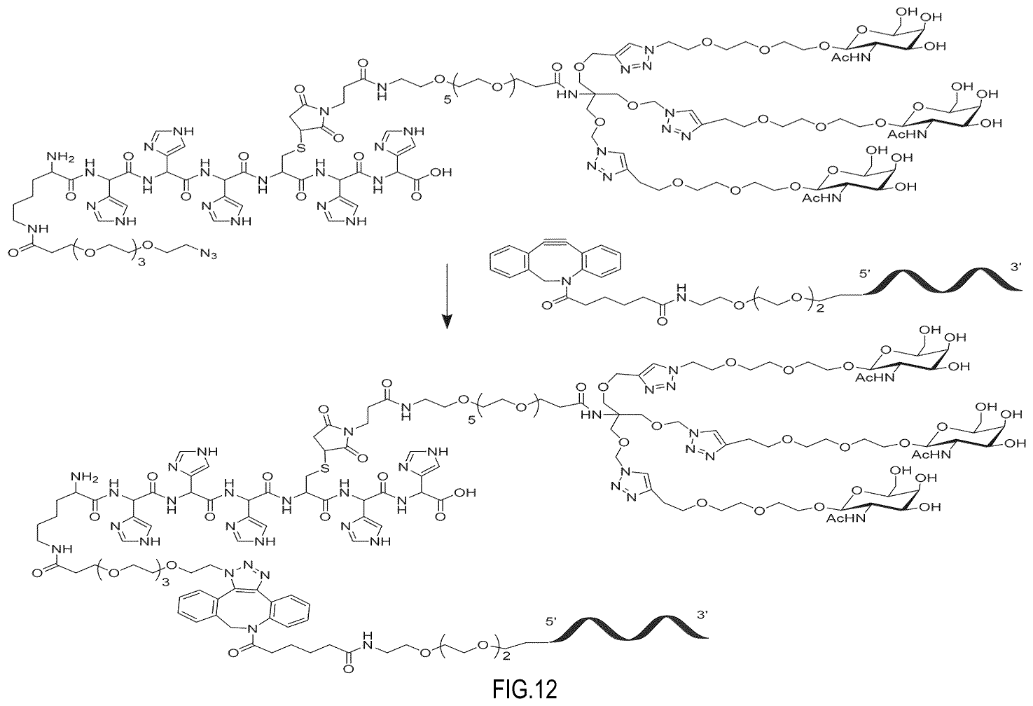

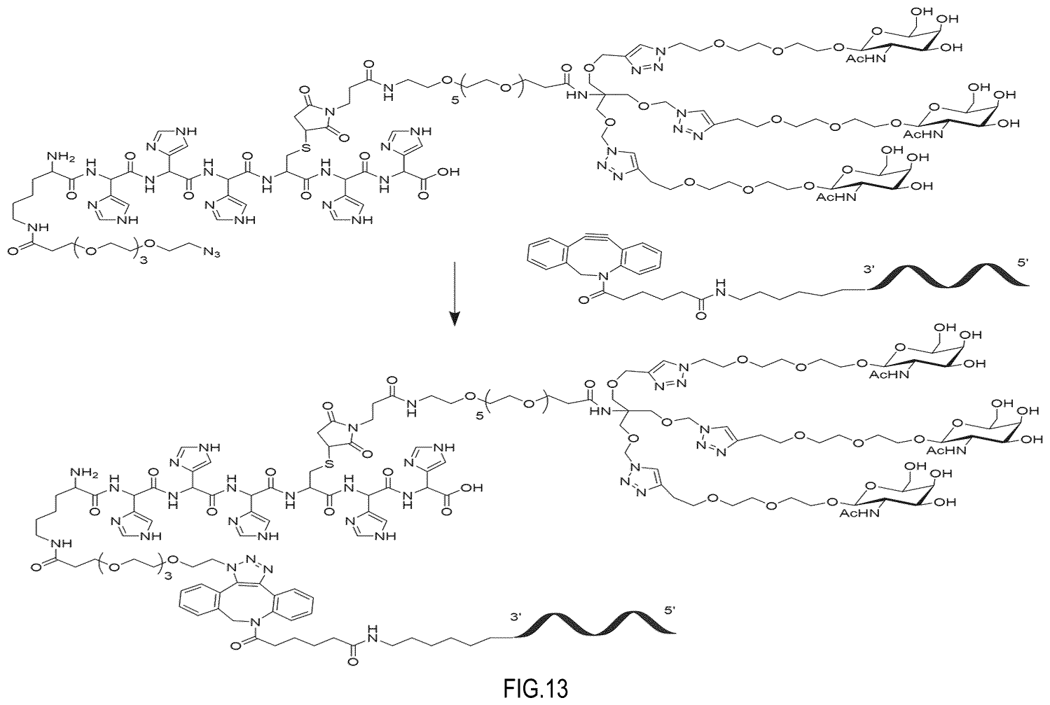

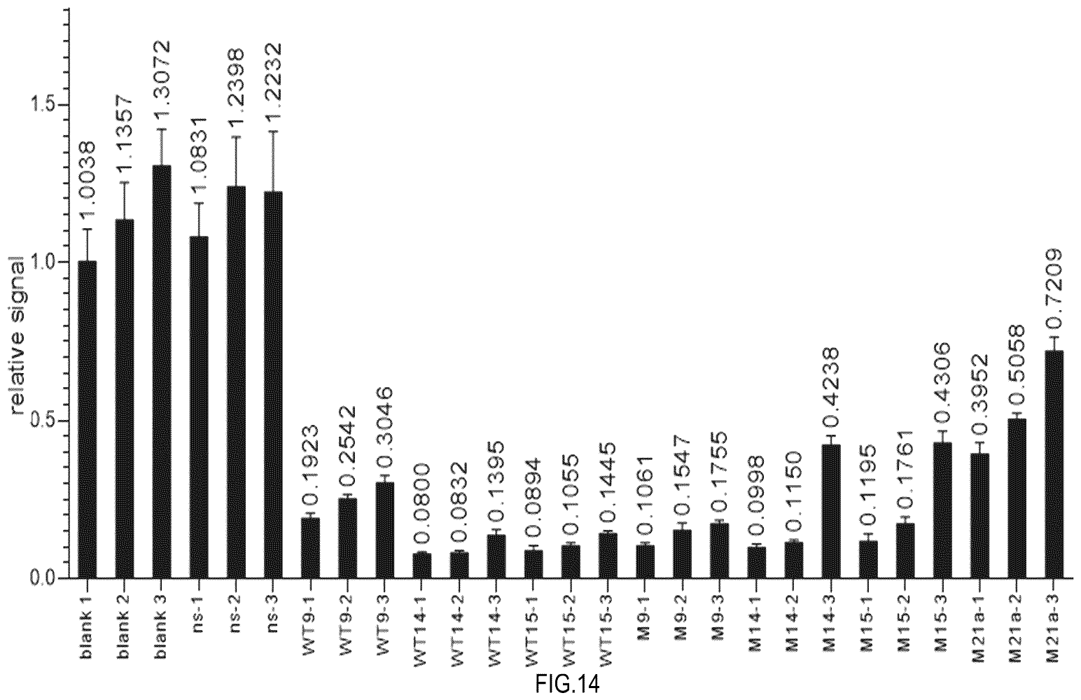

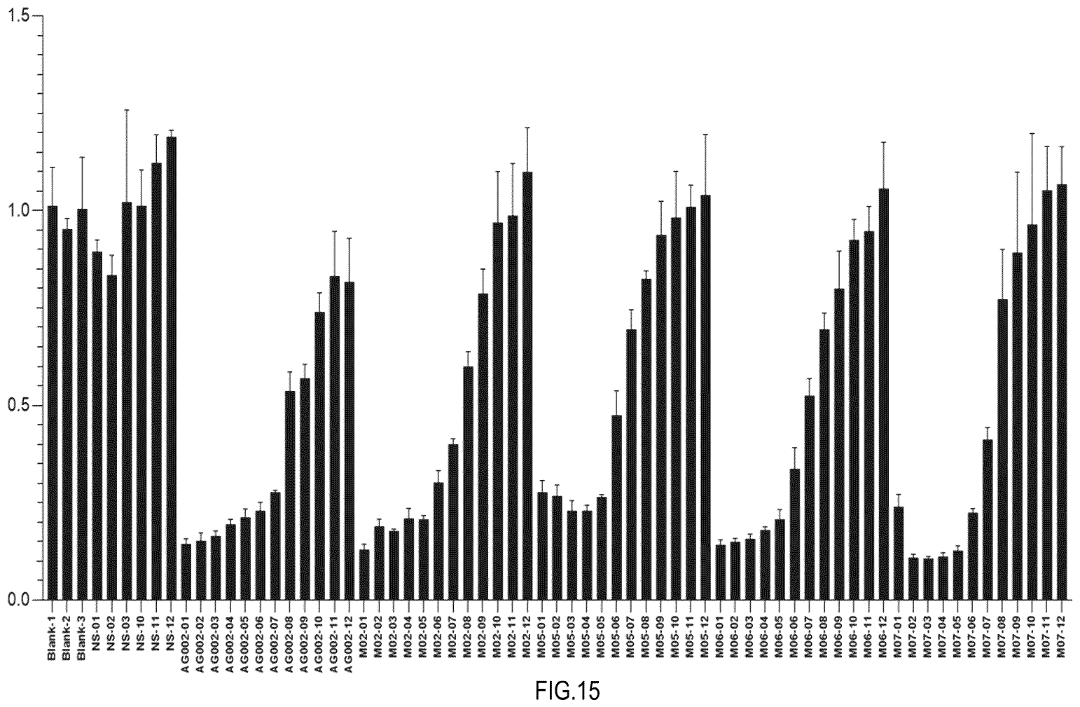

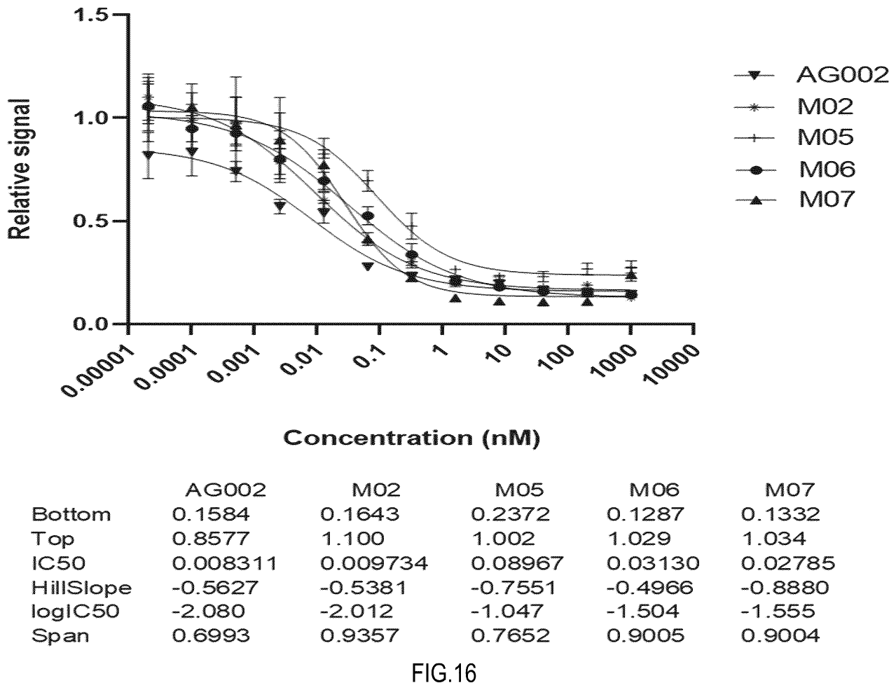

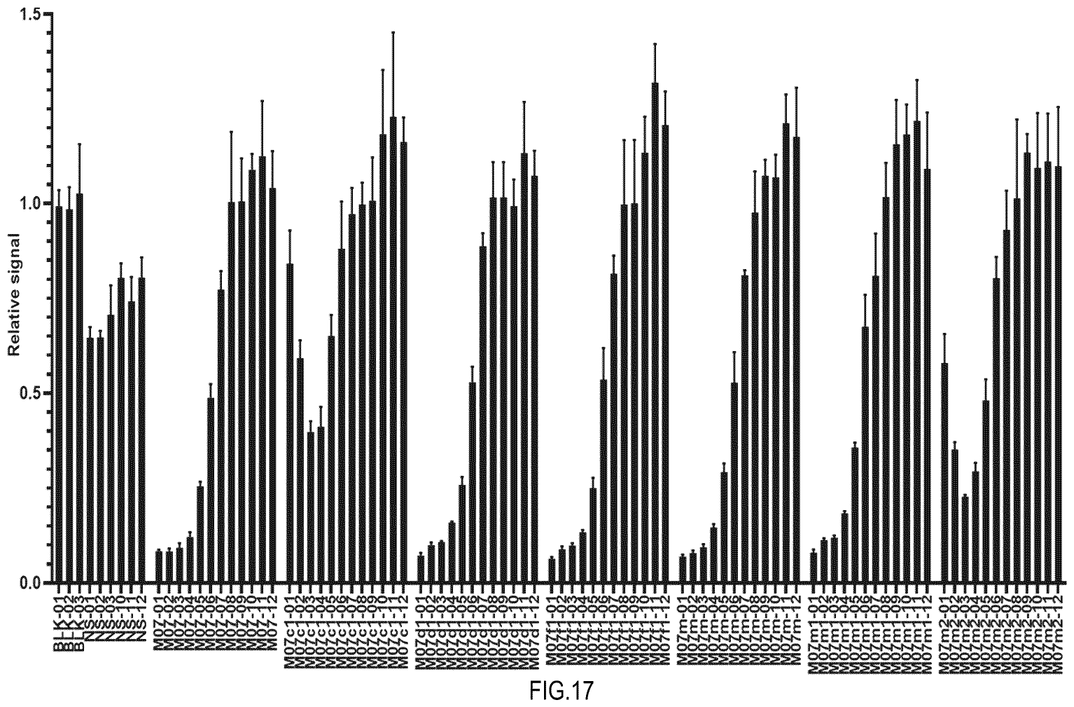

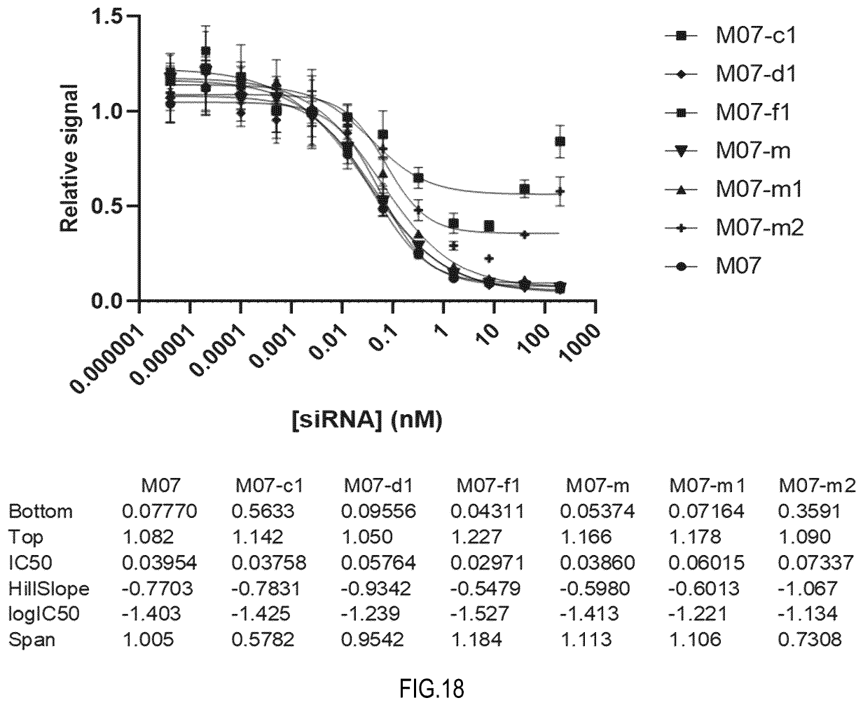

The Peptide Docking Vehicle (PDoV) advantageously has one site for conjugation to a targeting and at least one additional site for conjugation of an oligonucleotide. The PDoV has a peptide backbone with the general structure: (HₙKₘ)ₒXₚZ_{q} (SEQ ID NO: 132) with multiple repeating units of histidine (H), lysine (K) and functional units X and Z (where X or Z is an amino acid, or an amino acid derivative [see structure in FIG. 11], and where: n=1−10; m=1−10; o=1−10, p=1−5, and q=1−5. HK repeating units have been demonstrated to facilitate endosome release. The lysine residues or the functional unit(s) X may be used as docking sites for the conjugation of ligands and Z provides docking sites for the conjugation of oligonucleotide via a different covalent linkage.

The PDoV enhances the deployment of its macromolecular cargo into the cellular cytoplasm in a non-toxic manner. This allows effective delivery of, for example, RNAi therapeutics. The structure of an exemplary PDoV is shown in FIG. 11. The PDoV peptide acts both as the docking site linker for the RNA and the targeting ligands. The histidine and lysine rich polypeptide or linear histidine and lysine rich peptide has been shown to be an effective cell penetrating and endosomal release agent in the delivery of RNA. The PDoV peptide contains a histidine rich domain, where the imidazole rings of the histidine residues are protonated at a lower pH value (pH <˜6) and act inside the endosome as a proton sponge, which leads to lysis of the endosome lipid bilayers and release of the siRNA. The conjugation sites on the PDoV are described in more detail below (SEQ ID NOS 137-138, respectively, in order of appearance).

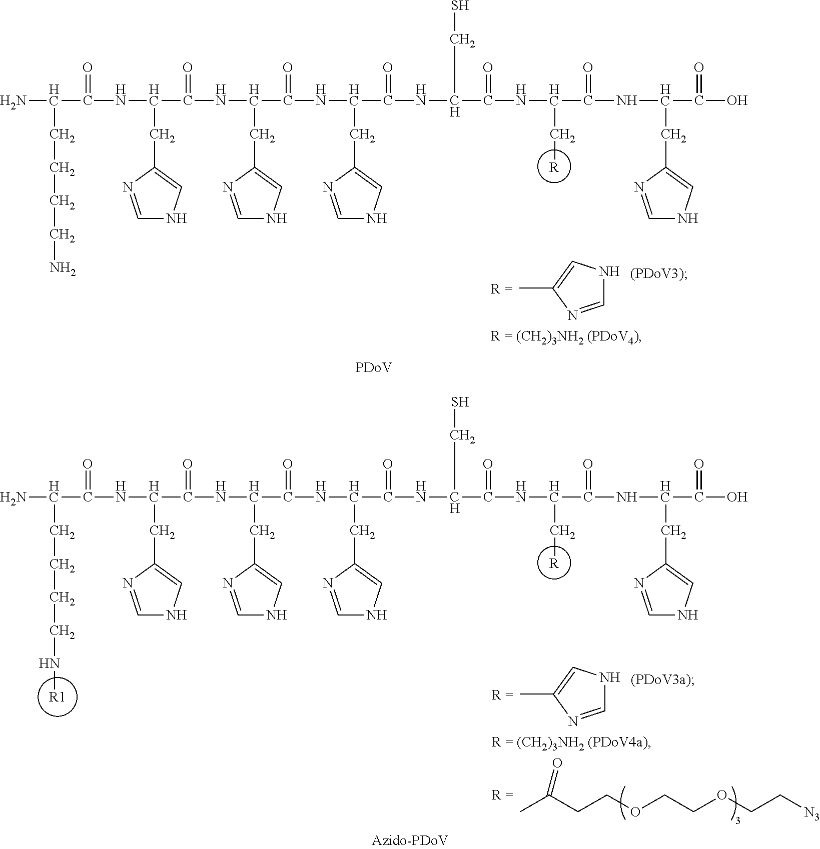

The oligonucleotide (RNA) sequences designed to target AGT mRNA are shown in Table 1. Specific modified sequences are shown in Table 2.

TABLE 1
Designed siRNA sequences

| SS | | | | | |
|---|---|---|---|---|---|
| SEQ | | | | | |
| ID | | Sequence | SS | AS | |
| No. | Name | Name | Sequence (5′ to 3′) | SEQ ID No. | Anti-Sense (5′ to 3′) |
| 1 | wt01 | AGT01 | GGGUGUACAUACACC | 23 | AAGGGGUGUAUGUACACCCdTdT |
| | | | CCUU | | |
| 2 | wt02 | AGT02 | GGGUGUACAUACACC | 24 | AAGUUUUGCAGCGACUAGCdTdT |
| | | | CCUU | | |
| 3 | wt03 | AGT03 | GCUUCCGUAUAUAUG | 25 | AUGCCAUAUAUACGGAAGCdTdT |
| | | | GCAU | | |
| 4 | wt04 | AGT04 | CCCUGGCUUUCAACA | 26 | UAGGUGUUGAAAGCCAGGGdTdT |
| | | | CCUA | | |
| 5 | wt05 | AGT05 | GGAGUGACAUCCAGG | 27 | UUGUCCUGGAUGUCACUCCdTdT |
| | | | ACAA | | |
| 6 | wt06 | AGT06 | GGGUCUCACUUUCCA | 28 | UUGCUGGAAAGUGAGACCCdTdT |
| | | | GCAA | | |
| 7 | wt07 | AGT07 | GGUCUCACUUUCCAG | 29 | UUUGCUGGAAAGUGAGACCdTdT |
| | | | CAAA | | |
| 8 | wt08 | AGT08 | GGGAGGUGCUGAACA | 30 | AUGCUGUUCAGCACCUCCCdTdT |
| | | | GCAU | | |
| 9 | wt09 | AGT09 | CCUGUUUGCUGUGUA | 31 | AUCAUACACAGCAAACAGGdTdT |
| | | | UGAU | | |
| 10 | wt10 | AGT10 | CCCCUGGCCUUUGAG | 32 | UUGCCUCAAAGGCCAGGGGdTdT |
| | | | GCAA | | |
| 11 | wt11 | AGT11 | CCCUGGCCUUUGAGG | 33 | UUUGCCUCAAAGGCCAGGGdTdT |
| | | | CAAA | | |
| 12 | wt12 | AGT12 | CCCCCAGUCUCCCACC | 34 | AAAGGUGGGAGACUGGGGGdTdT |
| | | | UUU | | |
| 13 | wt13 | AGT13 | CCUCCCAGUUUGCUG | 35 | AACCCAGCAAACUGGGAGGdTdT |
| | | | GGUU | | |
| 14 | wt14 | AGT14 | CCAGUUUGCUGGGUU | 36 | AAUAAACCCAGCAAACUGGdTdT |
| | | | UAUU | | |
| 15 | wt15 | AGT15 | CGGGACUACUGUUCC | 37 | UUUUGGAACAGUAGUCCCGdTdT |
| | | | AAAA | | |
| 16 | wt16 | AGT16 | CGACCAGCUUGUUUG | 38 | UUCACAAACAAGCUGGUCGdTdT |
| | | | UGAA | | |
| 17 | wt17 | AGT17 | GCAUUGCCUUCGGUU | 39 | UACAAACCGAAGGCAAUGCdTdT |
| | | | UGUA | | |
| 18 | wt18 | AGT18 | GGUUUGUAUUUAGUG | 40 | AAGACACUAAAUACAAACCdTdT |
| | | | UCUU | | |
| 19 | wt19 | AGT19 | GCUUGUGAUUUUUGA | 41 | UUGUUCAAAAAUCACAAGCdTdT |
| | | | ACAA | | |
| 20 | wt20 | AGT20 | GCGGAACCAUAGCUG | 42 | UAACCAGCUAUGGUUCCGCdTdT |
| | | | GUUA | | |
| 21 | wt21 | AGT21 | UCAUCCACAAUGAGA | 43 | UUACUCUCAUUGUGGAUGAdTdT |
| | | (Ag001) | GUAA | | |
| 22 | wt22 | AGT22 | GUCAUCCACAAUGAG | 44 | UGUACUCUCAUUGUGGAUGACGA |
| | | (Ag002) | AGUACA | | |

In some embodiments, the RNA also can be directly conjugated to a targeting ligand (for example N-acetyl-galactosamine), via, for example, the 3′ or 5′ terminal end of the RNA. In some embodiments, the RNA may contain one or more modified nucleotides such as 3′-OMe, 3′-F, or 3′-MOE. In some embodiments, the RNA can be an RNAi agent, for example a double stranded RNAi agent. In some embodiments, the targeting ligands disclosed herein are linked to the 5′ or 3′ terminus of the sense strand of a double stranded RNAi agent, or to the 5′ or 3′ terminus of the antisense strand of a double stranded RNAi agent. A major challenge for RNA-based therapeutics is that all pathways for delivery to cells must eventually lead to endosomal escape. ASO and siRNA delivery to the liver can be achieved using ASGPR-targeted GalNAc-siRNA conjugates due to the properties of ASGPR that are well suited for macromolecular drug deliver to hepatocytes.

Chemically modified siRNA sequences also were designed and tested for biological activities by in vitro assay using a variety of methods and technologies. These sequences are shown in Table 2 below. In some embodiments, the chemically modified siRNA sequences were chemically conjugated with a Peptide Docking Vehicle (PDoV) to improve delivery efficacy.

TABLE 2
Designed modified sequences

| SS | | | AS | |
|---|---|---|---|---|
| SEQ | Orig. | | SEQ | |
| ID | Sequence | SS | ID | |
| No. | Name | Sequence (5′ to 3′) | No. | Anti-Sense (5′ to 3′) |
| 45 | M02 | GmsCmsUmAmGmUmCfGfCfUmGm | 77 | PAmsAfsGmUmUmUmUmGmCmAm |
| | | CmAmAmAmAmCmUmUm | | GmCmGmAfCmUmAmGmsCmsdTdT |
| 46 | M04 | CmsCmsCmUmGmGmCfUfUfUmC | 78 | PUmsAfsGmGmUmGmUmUmGmAm |
| | | mAmAmCmAmCmCmUmAm | | AmAmGmCfCAGGsGsdTdT |
| 47 | M05 | GmsGmsAmGmUmGmAfCfAfUmC | 79 | PUmsUfsGmUmCmCmUmGmGmAm |
| | | mCmAmGmGmAmCmAmAm | | UmGmUmCfAmCmUmCmsCmsdTdT |
| 48 | M06 | GmsGmsGmUmCmUmCfAfCfUmU | 80 | PUmsUfsGmCmUmGmGmAmAmAm |
| | | mUmCmCmAmGmCmAmAm | | GmUmGmAfGmAmCmCmsCmsdTdT |
| 49 | M07 | GmsGmsUmCmUmCmAfCfUfUmUmC | 81 | PUmsUfsUmGmCmUmGmGmAmAm |
| | | mCmAmGmCmAmAmAm | | AmGmUmGfAmGmAmCmsCmsdTdT |
| 50 | M07c1 | GmsGmsUmCmUmCmAfCmUfUmUmCmCmA | 82 | PUmsUfsUmGmCmUmGmGmAmAmAmGmUm |
| | | mGmCmAmAmAm | | GfAmGmAmCmsCmsdTdT |
| 51 | M07d1 | GmsGmsUmCmUmCmAfCfUfUmUmCmCmAm | 83 | PUmsUfsUmGfCmUmGmGmAmAmAmGmUmGf |
| | | GmCmAmAmAm | | AmGmAmCmsCmsdTdT |
| 52 | M07f1 | GmsGmsUmCmUmCmAfCfUfUmUmCmCmAm | 84 | PUmsUfsUmGfCmUmGmGmAmAmAmGmUmGf |
| | | GmCmAmAmAm | | AmGfAmCmsCmsdTdT |
| 53 | M07m | GmsGmsUmCmUmCmAfCfUfUmUmCmCmAm | 85 | PUmsUfsUmGmCmUmGmGmAmAmAmGmdTGf |
| | | GmCmAmAmAm | | AmGmAmCmsCmsdTdT |
| 54 | M07m1 | GmsGmsUmCmUmCmGfCfUfUmUmCmCmAm | 86 | PUmsUfsUmGmCmUmGmGmAmAmAmGmdTGf |
| | | GmCmAmAmAm | | AmGmAmCmsCmsdTdT |
| 55 | M07m2 | GmsGmsUmCmUmCmAfCmUfUmUmCmCmA | 87 | PUmsUfsUmGmCmUmGmGmAmAmAmGmdTGf |
| | | mGmCmAmAmAm | | AmGmAmCmsCmsdTdT |
| 56 | M08 | GmsGmsGmAmGmGmUfGfCfUmG | 88 | PAmsUfsGmCmUmGmUmUmCmAmGmCmAmC |
| | | mAmAmCmAmGmCmAmUm | | fCmUmCmCmsCmsdTdT |
| 57 | M09 | CmsCmsUmGmUmUmUfGfCfUmGmUmGmUm | 89 | PAmsUfsCmAmUmAmCmAmCmAmGmCmAmA |
| | | AmUmGmAmUm | | fAmCmAmGmsGmsdTdT |
| 58 | M10 | CmsCmsCmCmUmGmGfCfCmUmUmUmGmA | 90 | PUmsUfsGmCmCmUmCmAmAmAGmGmCmCf |
| | | mGmGmCmAmAm | | AmGmGmGmsGmsdTdT |
| 59 | M11 | CmsCmsCmUmGmGmCfCfUfUmUmGmAmGm | 91 | PUmsUfsUmGmCmCmUmCmAmAmAmGmGmC |
| | | GmCmAmAmAm | | fCmAmGmGmsGmsdTdT |
| 60 | M12 | CmsCmsCmCmCmAmGfUfCfUmCmCmCmAm | 92 | PAmsAmsAmGmGmUmGmGmGmAmGmAmCm |
| | | CmCmUmUmUm | | UfGmGmGmGmsGmsdTdT |
| 61 | M13 | CmsCmsUmCmCmCmAfGfUfUmUmGmCmUm | 93 | PAmsAfsCmCCAGCAAACUGfGGAGGdTdT |
| | | GmGmGmUmUm | | |
| 62 | M14 | CmsCmsAmGmUmUmUfGfCfUmGmGmGmUm | 94 | PAmsAfsUmAmAmAmCmCmCmAmGmCmAmA |
| | | UmUmAmUmUm | | fAmCmUmGmsGmsdTdT |
| 63 | M15 | CmsGmsGmGmAmCmUfAfCfUmGmUmUmCm | 95 | PUmsUfsUmUmGmGmAmAmCmAmGmUmAm |
| | | CmAmAmAmAm | | GfUmCmCmCmsGmsdTdT |
| 64 | M16 | CmsGmsAmCmCmAmGfCfUfUmGmUmUmUm | 96 | PUmsUfsCmAmCmAmAmAmCmAmAmGmCmU |
| | | GmUmGmAmAm | | fGmGmUmCmsGmsdTdT |
| 65 | M21 | GmsUmsCmAmUmCmCfAmCfAfAfUmGmAmG | 97 | PUmsGfsUmAmCmUmCmUmCmAmUmUmGmU |
| | | mAmGmUmAmCmAm | | fGmGfAmUmGmAmCmsGmsAm |
| 66 | M21a | GmsUmsCmAmUmCmCfAfCfAmAmUmGmAm | 98 | PUmsGfsUmAmCmUmCmUmCmAmUmUmGmU |
| | | GmAmGmUmAmCmAm | | fGmGmAmUmGmAmCmsGmsAm |
| 67 | M21b | GmsUmsCmAmUmCmCfAmCfAfAfUmGmAmG | 99 | PUmsGfsUmAmCmUmCmUmCmAmUmUmGmU |
| | | mAmGmUmAmCmAm | | fGmGfAmUmGmAmCmsGmsAm |
| 68 | M21c1 | GmsUmsCmAmUmCmCfAmCfAmAmUmGmA | 100 | PUmsGfsUmAmCmUmCmUmCmAmUmUmGmU |
| | | mGmAmGmUmAmCmAm | | fGmGmAmUmGmAmCmsGmsAm |
| 69 | M21d1 | GmsUmsCmAmUmCmCfAmCfAmAmUmGmA | 101 | PUmsGfsUmAfCmUmCmUmCmAmUmUmGmUf |
| | | mGmAmGmUmAmCmAm | | GmGmAmUmGmAmCmsGmsAm |
| 70 | M21m | GmsUmsCmAmUmCmCfAmCfAmAmUmGmA | 102 | PUmsGfsUmAfCmUmCmUmCmAmUmUmGmUf |
| | | mGmAmGmUmAmCmAm | | GmGfAmUmGmAmCmsGmsAm |
| 71 | APG3M06 | GalNAc-PDoV3-GmsGmsGmUmCmUmCfAf | 103 | PUmsUfsGmCmUmGmGmAmAmAmGmUmGm |
| | | CfUmUmUmCmCmAmGmCmAmAm | | AfGmAmCmCmsCmsdTdT |
| 72 | APG3M07 | GalNAc-PDoV3-GmsGmsUmCmUmCm | 104 | PUmsUfsUmGmCmUmGmGmAmAmAmGmUm |
| | | AfCfUfUmUmCmCmAmGmCmAmAmAm | | GfAmGmAmCmsCmsdTdT |
| 73 | APG3M07a | GmsGmsUmCmUmCmAfCfUfUmUmCmCmAm | 105 | PUmsUfsUmGmCmUmGmGmAmAmAmGmUm |
| | | GmCmAmAmAm-PDoV3-GalNAc | | GfAmGmAmCmsCmsdTdT |
| 74 | APG3M14 | GalNAc-PDoV3-CmsCmsAmGmUmUm | 106 | PAmsAfsUmAmAmAmCmCmCmAmGmCmAmA |
| | | UfGfCfUmGmGmGmUmUmUmAmUmUm | | fAmCmUmGmsGmsdTdT |
| 75 | APG3M21 | GalNAc-PDoV3-GmsUmsCmAmUmCmCf | 107 | PUmsGfsUmAmCmUmCmUmCmAmUmUmGmU |
| | | AmCfAfAfUmGmAmGmAmGmUmAmCmAm | | fGmGfAmUmGmAmCmsGmsAm |
| 76 | APGM21a | GalNAc-PDoV3-GmsUmsCmAmUmCmCfAf | 108 | PUmsGfsUmAmCmUmCmUmCmAmUmUmGmU |
| | | CfAmAmUmGmAmGmAmGmUmAmCmAm | | fGmGmAmUmGmAmCmsGmsAm |

Note that in the table (2) immediately above, there are 2′-modifications in all sequences, with the following designations: upper case X = normal 2′-OH nucleotide; Xm = indicates 2′-O-methyl(2′-OMe) modification; Xf-2′-F modification; dT = deoxythymidine; P = phosphate; s = phosphorothioate (PS).

### Determination of Efficacy of the siRNA Molecules

Depending on the target mRNA sequences and the dose of the nanoparticle composition delivered, partial or complete loss of function for the target mRNAs may be observed. A reduction or loss of mRNA levels, gene expression or encoded polypeptide expression in at least 10%, at least 20%, at least 30%, at least 40%, at least 50%, at least 60%, at least 70%, at least 80%, at least 90%, at least 95% or at least 99% or more of targeted cells is exemplary. Inhibition of targeted mRNA levels or gene expression refers to the absence (or observable decrease) in the level of the target mNA or the RNA-encoded peptide or protein. Specificity refers to the ability to inhibit the target mRNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (MA), other immunoassays, and fluorescence activated cell analysis (FACS). Inhibition of target mRNA sequence(s) by the dsRNA agents in the disclosed embodiments also can be measured based upon the effect of administration of such dsRNA agents upon development/progression of a target mRNA-associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject. The subject may be a mammal, such as a human.

### Determination of Dosage and Toxicity

Toxicity and therapeutic efficacy of the compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds advantageously exhibit high therapeutic indices.

Data from cell culture assays and animal studies can be used in formulating a range of dosage for use in humans. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ (i.e., the concentration of the composition which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a composition as described herein can be in the range of approximately 1 pg to 1000 mg. For example, 10, 30, 100, or 1000 pg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30 percent reduction in a measurable parameter associated with a disease or disorder, a therapeutically effective amount of a drug for the treatment of that disease or disorder is the amount necessary to effect at least a 30 percent reduction in that parameter.

### Histidine-lysine Copolymer Carrier Delivery of siRNA to Target AGT

Also provided are pharmaceutical compositions comprising the anti-AGT siRNA PDoV conjugate and a pharmaceutically acceptable copolymer carrier for in vitro and in vivo delivery to gene targets. The pharmaceutically acceptable carrier may contain a histidine-lysine rich polypeptide (HKP, HKP(+H)), or lipofectamine, and/or may contain water and one or more of the group consisting of: potassium phosphate monobasic anhydrous NF, sodium chloride USP, sodium phosphate dibasic heptahydrate USP, glucose, and Phosphate Buffered Saline (PBS). Histidine-lysine copolymer carriers are described in more detail in U.S. Pat. Nos. 7,163,295, 7,070,807, and 7,772,201, and in U.S. patent application Ser. No. 17/713,037. Suitable HKP carriers include H3K4b and H3K4b(+H) as described in more detail in these patents and patent applications.

In still other aspects, the PDoV conjugate and the HKP are formulated into nanoparticles using methods known in the art. See, for example, Babu et al., IEEE Trans Nanobioscience, 15: 849-863 (2016). HKP copolymers form a nanoparticle containing an siRNA molecule, typically 100-400 nm in diameter. HKP and HKP(+H) both have a lysine backbone (three lysine residues) where the lysine side chain c-amino groups and the N-terminus are coupled to [KH₃]₄K (SEQ ID NO: 133) (for HKP) or KH₃KH₄[KH₃]₂K (SEQ ID NO: 134) (for HKP(+H). The branched HKP carriers can be synthesized by methods that are well-known in the art including, for example, solid-phase peptide synthesis.

Methods of forming nanoparticles are well known in the art. Babu et al., supra.

Advantageously, nanoparticles may be formed using a microfluidic mixer system, in which a PDoV conjugate containing an siRNA targeting AGT is mixed with one or more HKP polymers at a fixed flow rate. The flow rate can be varied to vary the size of the nanoparticles produced. A suitable mixer is a PNI microfluidic mixer system (Precision Nanosystems, Inc., Vancouver, CA). The Total Flow Rate (TFR) may be varied and the effect of this flow rate on particle size is evaluated by measuring resulting particle size using a Malvern Nanosizer system (Malvern Panalytical Inc., Westborough, MA). The polydispersity index (PDI) is an indication of the amount of variation of the nanoparticles around the average size.

### Pharmaceutical Compositions and Methods of Administration and Treatment

The siRNA compositions comprising at least one siRNA linked to PDoV may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Advantageously, the pharmaceutical compositions are administered by intravenous or intraparenteral infusion or injection.

For transmucosal or transdermal administration, penetrants appropriate to the barrier to be permeated are used in the formulation. Such penetrants are generally known in the art, and include, for example, for transmucosal administration, detergents, bile salts and fusidic acid derivatives. Transmucosal administration can be accomplished through the use of nasal sprays or suppositories. For transdermal administration, the active compounds are formulated into ointments, salves, gels, or creams, and applied through dermal patches and the lie, as generally known in the art.

The siRNA formulations can also be administered by transfection or infection using methods known in the art, including but not limited to the methods described in McCaffrey et al. (2002), Nature, 418(6893), 38-9 (hydroydynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996). Further, the siRNA formulations can also be administered by a method suitable for administration of nucleic acid agents, such as a DNA vaccine. These methods include gene guns, bio-injectors, and skin patches as well as needle-free methods such as the micro-particle DNA vaccine technology disclosed in the U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al (1998), Clin. Immunol. Immunopath., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that it may be administered through a syringe or similar device. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition.

Prolonged absorption of the injectable compositions can be brought about by including in the composition an agent which delays absorption, for example, aluminum monostearate and gelatin. Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The oligonucleotides and pharmaceutical compositions described herein are designed to treat hypertension by targeting AGT, one of the key components in the RAAS. Likewise, the oligonucleotide-PDoV complex may be used to treat any number or variety of diseases or disorders by targeting one or more other peptides or proteins known to have a role in initiation or progression of the disease(s), such as cancers and fibrosis, for example. The composition may be delivered, for example, systemically or intratumorally.

The compositions may be administered as a monotherapy, i.e. in the absence of another treatment, or may be administered as part of a combination regimen that includes one or more additional medications. Advantageously, the compositions are used as part of a combination regimen that includes an effective amount of at least one additional active pharmaceutical agent.

The disclosed embodiments are further illustrated by the examples below, which are non-limiting.

### EXAMPLES

### Example 1. Transfection in HepG2 Cells

1×10⁵ HepG2 cells in 0.5 ml DMEM supplemented with 10% fetal bovine serum (FBS) (Gibco) per well were seeded into 24-well plates. 24 hours after seeding, cells were transfected with siRNA by mixing siRNA with 1.5 μl lipofectamine RNAi max (ThermoFisher) and 100 μl OptiMEM media (Gibco) per sample for 15 minutes, then adding the mixture to HepG2 cells. Cells were lysed and RNA was harvested 48 hrs post-transfection using the RNeasy plus mini kit (Qiagen).

### Example 2. Transfection in Primary Human Hepatocytes

Frozen primary human hepatocytes (Xenotech) were thawed in hepatocyte thawing media (xenotech). Once thawed, cells were centrifuged for 5 minutes at 100 g and resuspended in William's E medium (WEM) supplemented with Primary Hepatocyte Thawing and Plating Supplements (Gibco), referred to as WEM complete. 1×10⁵ hepatocytes in 0.450 mls WEM complete per well were seeded into 24 well collagen I coated 24 plates (Stemcell technologies). After 5 hours of incubation at 37° C., siRNA diluted into 50 μl basal WEM was added to wells. Cells were lysed and RNA was harvested 72 hrs post-transfection using the RNeasy plus mini kit (Qiagen).

### Example 3. cDNA Synthesis

cDNA was synthesized using the Maxima First Strand Synthesis kit (ThermoFisher). RNA was incubated with buffer and enzyme for at 25° C. for 10 minutes, 50° C. for 15 minutes, and 85° C. for 5 minutes.

### Example 4. Quantitative Real-time PCR (qPCR)

qPCR was performed using TaqPath master mix (Applied Biosystems) and human AGT primer and Taqman probe set (Thermofisher). Hprt1 was used as an endogenous control in HepG2 cells. Gapdh was used as an endogenous control for primary human hepatocytes. A QuantStudio 5 real-time PCR machine (Thermofisher) and a program of 50° C. for 2 minutes, 95° C. for 10 minutes followed by 95° C. for 15 minutes and 60° C. for 1 minute, repeated 40 times, and used for PCR amplification. Relative expression was calculated from delta-delta Ct values.

### Example 5. In Vitro Screening of the AGT siRNA Sequence

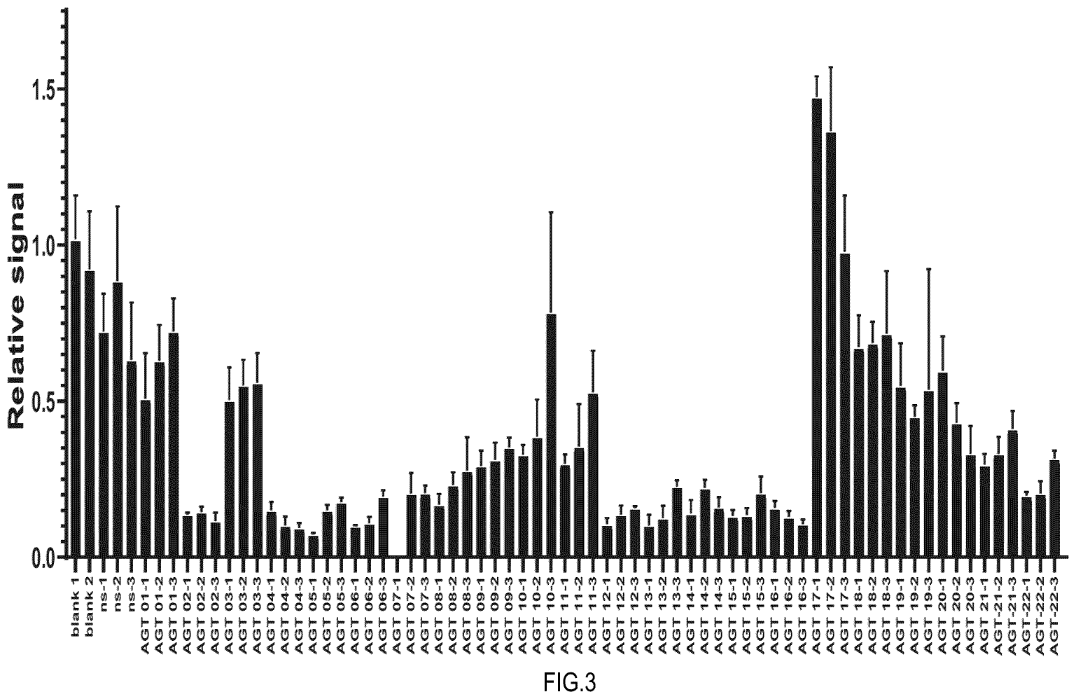

Knockdown efficacy study of unmodified AGT siRNA. The in vitro experiment was done in HepG2 cells, 1×10⁵ per well in 24-well plates, siRNA final concentration is 50 (1), 5 (2), and 0.5 (3) μM. Transfection duration was 48 hr. There are 22 AGT siRNA samples, plus NS control and, untreated cells (blank) in this setup. QRTPCR: HPRT (as internal control) and AGT primers were used at a final concentration of 160 and 320 nM, respectively. Taqman probes for each gene were used at a concentration of 200 nM per reaction. Most of AGT siRNA showed significant silencing comparing to Lipo NS except AGT01, AGT03, AGT17, AGt18. See FIG. 3. Excluding the previously mentioned inefficient siRNAs, the mRNA knockdown level is all greater than 74% and up to 94%. In case of AGT05, 06, 12, 13, 14, 15 the remaining AGT expression level is below 11%. Most of the designed siRNA showed great potency in the mRNA knockdown evaluation experiment.

### Example 6. In Vitro Screening of the AGT siRNA Sequence

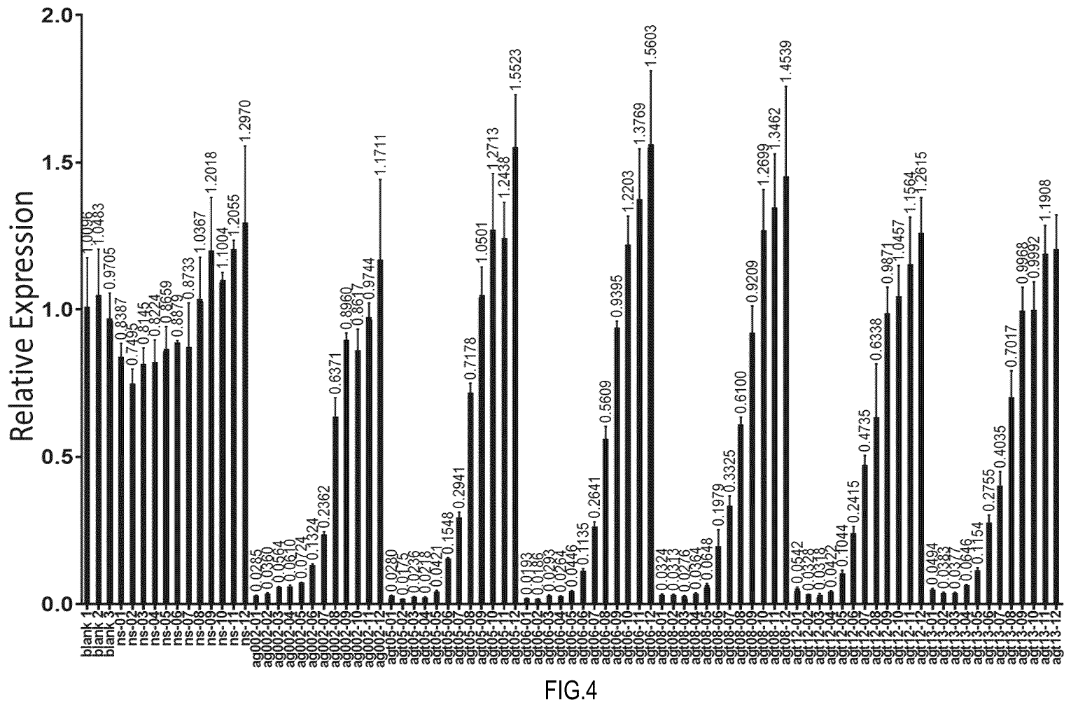

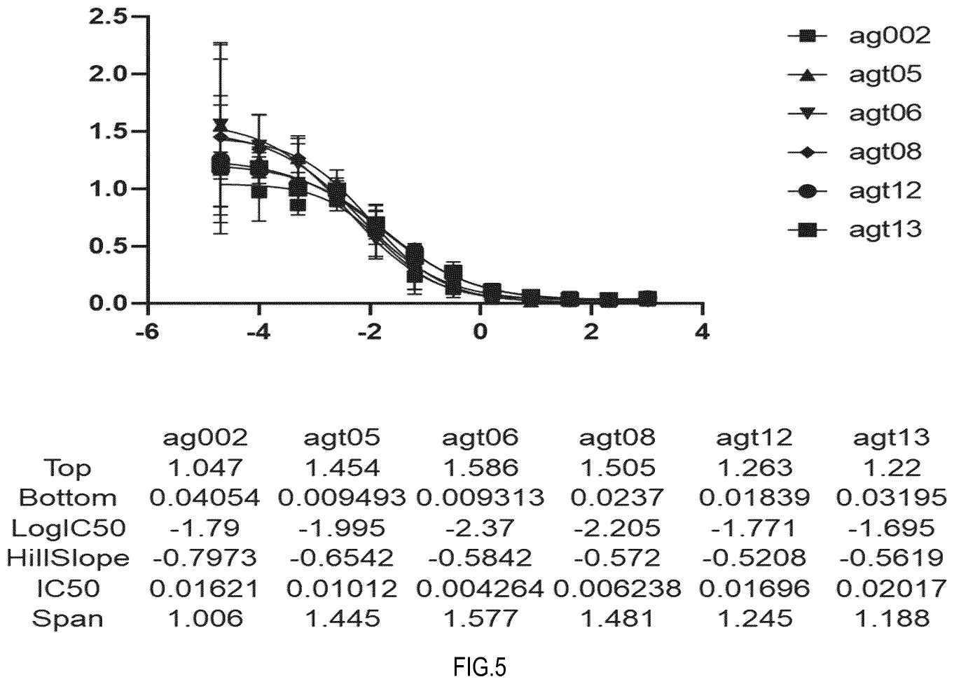

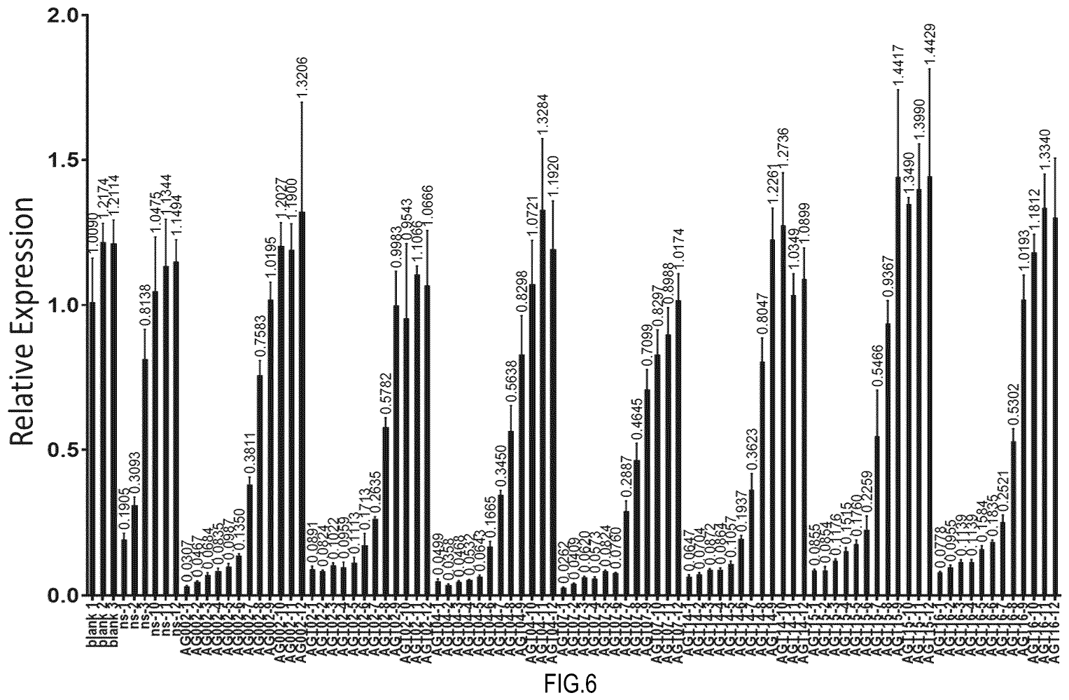

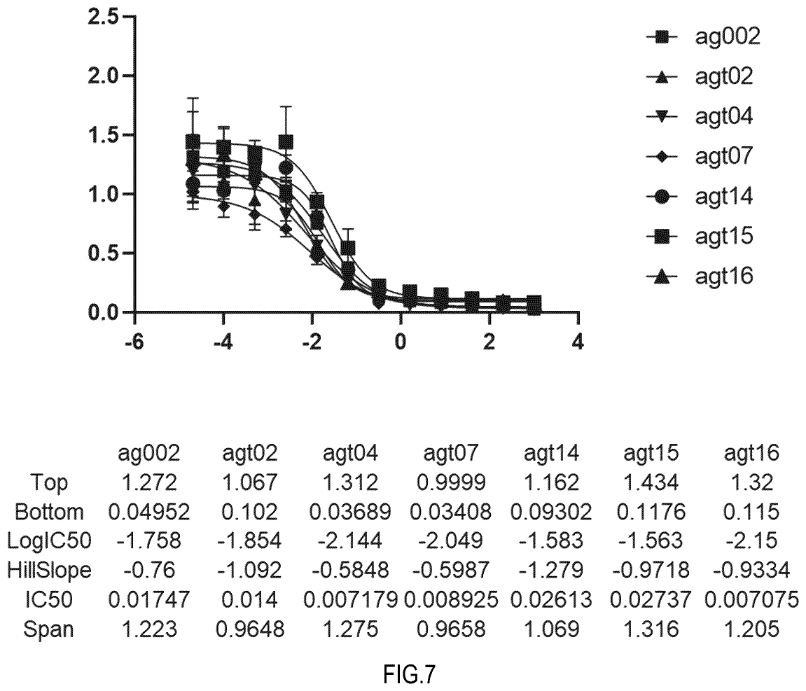

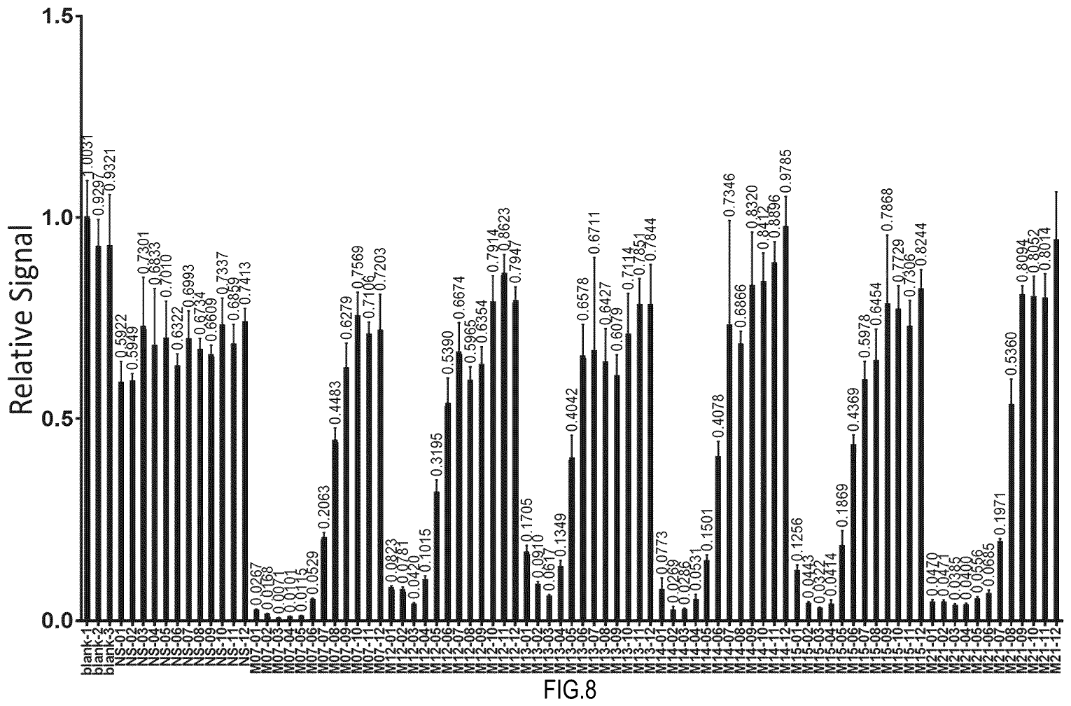

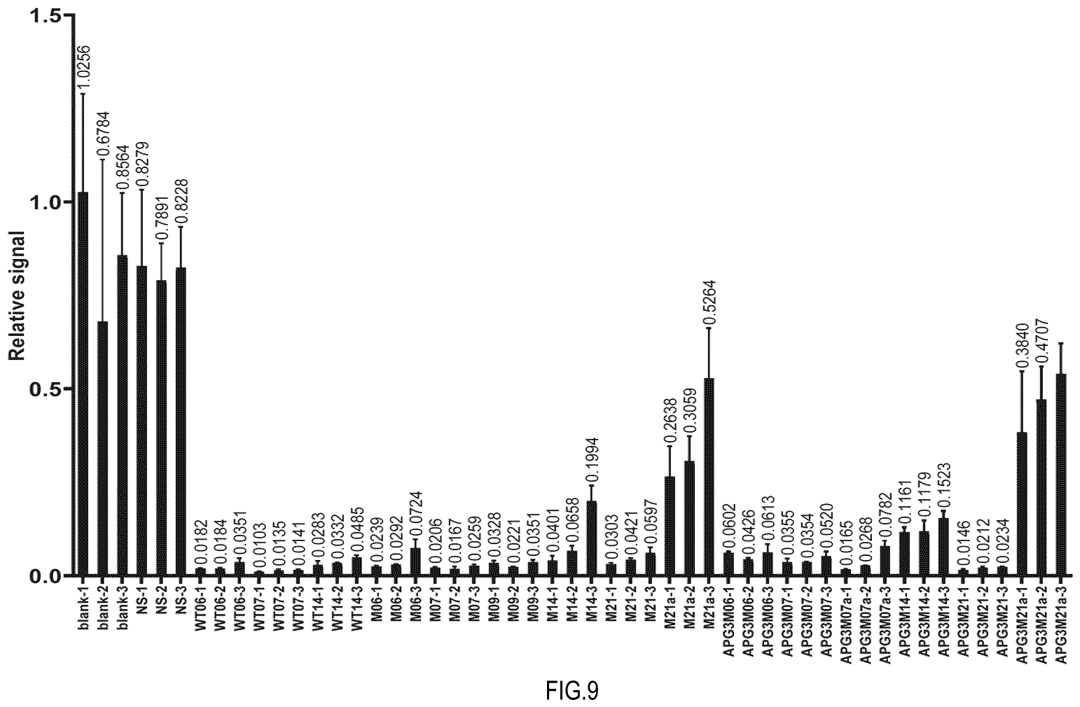

In vitro screening of the modified AGT siRNA sequence. HepG2: 2×10⁵ cells per well in 24-well plate in transfection time for 48 hr. cDNA was 25 ng per 10 μL PCR reaction. AGT siRNA concentrations in transfection: 1000, 200, 40, 8, 1.6, 0.32, 0.064, 0.0128, 0.00256, 0.000512, 0.0001024, and 0.00002048 nM, 5× serial dilution. Lipofectamine RNAi Max (1.5 μL/Rxn) and siRNA were mixed in 100 μL of OptiMEM and incubated for 15 min. Multiplex PCR was HPRT (as internal control) and AGT primers used at a final concentration of 160 and 320 nM, respectively. Taqman probes for each gene were used at a concentration of 200 nM per reaction. TaqPath 2× master mix was used as 5 μL per 10 μL reaction (see FIG. 4-FIG. 8). The siRNA was further chemically modified to enhance the stabilization. The mRNA knockdown level of those modified mAGT siRNA were further evaluated by serial dilution experiment. The IC50 values of AGT siRNA was calculated in Prism GraphPad. To generate IC₅₀ values, siRNA concentrations were transformed to the log₁₀ scale and the data were fitted to a sigmoidal four-parameter logistic (4PL) curve.

### Example 7. In Vitro Screening of the AGT siRNA Sequence in Human Primary Hepatocytes

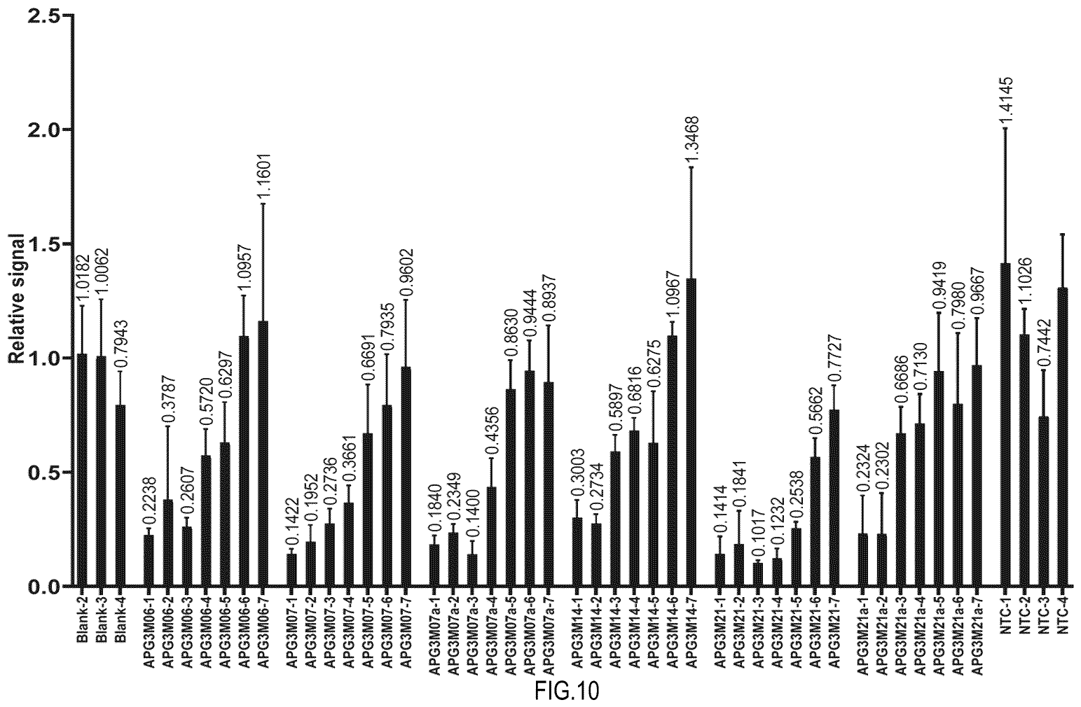

In vitro screening of the modified AGT siRNA sequence. Human primary hepatocytes: 1×10⁵ cells per well in 24-well plate in transfection time for 72 hr. cDNA was 25 ng per 10 μL PCR reaction. Start from 1 μM, 5× dilution, 7 conc point, siRNA. Thaw hepatocytes in thawing medium. Spin down, resuspend in WEM complete. Seed 450 μL hepatocytes on collagen coated plates, let them attach for 5 hours. Mix siRNA with basal WEM (50 μl). Add to hepatocytes. Multiplex PCR was Gapdh (internal control) and AGT primer and probe Taqman assay (20×, 0.5 μL each assay per 10 μL reaction). TaqPath 2× master mix was used as 5 μL per 10 μL reaction (see FIG. 10). The siRNA was further chemically modified to enhance the stabilization. The mRNA knockdown level of those modified AGT siRNA were evaluated by serial dilution experiment.

### Example 8. Synthesis of Oligonucleotides

Sense and antisense strands were synthesized on an Mermade12 Synthesizer (BioAutomation, Irving, Texas) using commercially available 5′-Dimethoxytrityl-N-isobutyryl-Guanosine,2′-O-methyl,3′-[(2-cyanoethyl)-(N,N-diisopropyl)]-phosphoramidite, 5′-Dimethoxytrityl-N-acetyl-Cytidine,2′-O-methyl,3′-[(2-cyanoethyl)-(N,N-diisopropyl)]-phosphoramidite, 5′-Dimethoxytrityl-N-benzoyl-Adenosine,2′-O-methyl,3′-[(2-cyanoethyl)-(N,N-diisopropyl)]-phosphoramidite, 5′-Dimethoxytrityl-Uridine,2′-O-methyl,3′-[(2-cyanoethyl)-(N,N-diisopropyl)]-phosphoramidite, 5′-Dimethoxytrityl-deoxyUridine, 2′-fluoro-3′-[(2-cyanoethyl)-(N,N-diisopropyl)]-phosphoramidite, 5′-Dimethoxytrityl-N-benzoyl-deoxyAdenosine, 2′-fluoro-3′-[(2-cyanoethyl)-(N,N-diisopropyl)]-phosphoramidite, 5′-Dimethoxytrityl-N-acetyl-deoxyCytidine,2′-fluoro-3′-[(2-cyanoethyl)-(N,N-diisopropyl)]-phosphoramidite, 5′-Dimethoxytrityl-N-isobutyryl-deoxyGuanosine, 2′-fluoro-3′-[(2-cyanoethyl)-(N,N-diisopropyl)]-phosphoramidite, 10-(6-oxo-6-(dibenzo[b,f]azacyclooct-4-yn-l-yl)-capramido-N-ethyl)-O-triethyleneglycol-1-[(2-cyanoethyl)-(N,N-diisopropyl)]-phosphoramidite with standard solid-phase oligonucleotide synthesis and deprotection protocols. The selection of solid support for each synthesis yielded the corresponding 3′-C6-NH₂ modified, 5′-dibenzocyclooctyl-TEG (5′-DBCO-TEG) modified or unmodified oligonucleotides. After synthesis, the support was treated with AMA (ammonium hydroxide/40% aqueous methylamine 1:1 v/v) at room temperature for 2 h. The suspension was centrifugated to remove solid residues, then was desalted and purified by Glen Gel-Pak™ 1.0 or 2.5 Desalting Column. The integrities of the purified oligonucleotides were confirmed by LC-MS and analytical RP-HPLC (Agilent AdvanceBio Oligonucleotide, 4.6×150 mm, 0.6 mL/min, 100 mM TEAA pH7.0/100 mM TEAA pH7.0+90% ACN).

TABLE 3
SS and AS sequences resulting from synthesis described in Example 8

| | | Sequence (5′-3′), (All of the ribose are | | | |
|---|---|---|---|---|---|
| SEQ | Orig. | 2′-OMe, except f is 2′-F modification. S | | | HPLC |
| ID | Seq. | is phosphorothioated modification. P is | Mass. | Mass. | purity |
| No. | Name | phospholation) | (Calc). | (Found) | (figure) |
| 109 | M06 | SS: DBCO-teg-GGGUCUCfAfCfUUUCCAGCsAsA | 6830.31 | 6830 | >90% |
| 110 | | AS: PUsUfsGCUGGAAAGUGAfGACCsCsdTdT | 7095.12 | 7095 | >90% |
| 111 | M07 | SS: DBCO-teg-GGUCUCAfCfUfUUCCAGCAsAsA | 6814.3 | 6814 | >90% |
| 112 | | AS: PUsUfsUGCUGGAAAGUGfAGACsCsdTdT | 7096.11 | 7096 | >85% |
| 113 | M07a | SS: GsGsUCUCAfCfUfUUCCAGCAAA-C6-NH2 | 6422.9 | 6423 | >90% |
| 114 | | AS: PUsUfsUGCUGGAAAGUGfAGACsCsdTdT | 7096.11 | 7096 | >90% |
| 115 | M14 | SS: DBCO-teg-CCAGUUUfGfCfUGGGUUUAsUsU | 6826.23 | 6826 | >90% |
| 116 | | AS: PAsAfsUAAACCCAGCAAfACUGsGsdTdT | 7069.18 | 7069 | >90% |
| 117 | M21 | SS: DBCO-teg- | 7551.79 | 7551 | >90% |
| | | GUCAUCCfACfAfAfUGAGAGUAsCsA | | | |
| 118 | | AS: PUsGfsUACUCUCAUUGUfGGfAUGACsGsA | 7755.48 | 7755 | >90% |
| 119 | M21a | SS: DBCO-teg- | 7563.81 | 7563 | >90% |
| | | GUCAUCCfAfCfAAUGAGAGUAsCsA | | | |
| 120 | | AS: PUsGfsUACUCUCAUUGUfGGAUGACsGsA | 7767.50 | 7767 | >90% |

### Example 9. Synthesis of 5′-GaINAc-PDoV-sense Strand Oligonucleotide

5′-DBCO-TEG-sense strand oligonucleotide was dissolved in deionized water to prepare the 0.5 mM solution. 1.05 equivalent amounts of GalNAc-PDoV solution (1 mM) was added into 0.5 ml 5′-DBCO-TEG-oligonucleotide solution, then this mixture was shaken slowly at room temperature for 1 h. After reaction, a 3 k cut-off centrifugal filter was applied to remove the excess GalNAc-PDoV to provide the purified 5′-GalNAc-PDoV-DBCO-TEG-sense strand oligonucleotide in 99% yield. Products were characterized and confirmed by HPLC (Agilent AdvanceBio Oligonucleotide, 4.6×150 mm, 0.6 mL/min, 100 mM TEAA pH7.0/100 mM TEAA pH7.0+90% ACN) and LC-MS.

TABLE 4
Synthesis with GalNAc-PDOV at 5′ end

| SEQ | Orig. | Sequence (5′-3′), (All of the riboses | | | |
|---|---|---|---|---|---|
| ID | Seq. | are 2′-OMe, except f is 2′-F modification. | Mass | Mass | HPLC |
| No. | Name | S is PS modification.) | (Calc) | (Found) | purity |
| 121 | APG3M06 | SS: GalNAc-PDoV3-DBCO-teg- | 9964.81 | 9967 | >90% |
| | | GGGUCUCfAfCfUUUCCAGCsAsA | | | |
| 122 | APG3M07 | SS: GalNAc-PDoV3DBCO-teg- | 9948.81 | 9951 | >90% |
| | | GGUCUCAfCfUfUUCCAGCASASA | | | |
| 123 | APG3M14 | SS: GalNAc-PDoV3DBCO-teg- | 9960.70 | 9963 | >90% |
| | | CCAGUUUfGfCfUGGGUUUAsUSU | | | |
| 124 | APG3M21 | SS: GalNAc-PDoV3DBCO-teg- | 10686.29 | 10688 | >90% |
| | | GUCAUCCfACfAfAfUGAGAGUAsCsA | | | |
| 125 | APGM21a | SS: GalNAc-PDoV3DBCO-teg- | 10698.21 | 10700 | >90% |
| | | GUCAUCCfAfCfAAUGAGAGUAsCsA | | | |

### Example 10. Synthesis of 3′-DBCO-NH-C6-sense Strand Oligonucleotide

3′-NH2-C6-sense strand oligonucleotide was dissolved in deionized water to prepare the 0.5 mM NaHCO₃ solution (50 mM pH9.0). Then 60 equivalents of DBCO-sulfo-NHS Ester was dissolved in 50 μL anhydrous DMSO and was added to oligonucleotide solution in 20 mins with stirring. After 3 h reaction, the solution passed Glen Gel-Pak™ desalting column to remove the small molecules and salts in reaction. RP-HPLC was applied to purify the crude products. Purification afforded 3′-DBCO-NH-C6-sense strand oligonucleotides in 90% yield. Products were characterized and confirmed by HPLC (Agilent AdvanceBio Oligonucleotide, 4.6×150 mm, 0.6 mL/min, 100 mM TEAA pH7.0/100 mM TEAA pH7.0+90% ACN) and LC-MS.

TABLE 5
Synthesis with DBCO-NH-C6

| | Orig. | Sequence (5′-3′), (All of the riboses | | | |
|---|---|---|---|---|---|
| SEQ | Seq. | are 2′-OMe, except f is 2′-F | Mass | Mass | HPLC |
| ID No. | name | modification. S is PS modification.) | (Calc) | (Found) | purity |
| 126 | M07a- | SS: | 6739.3 | 6738 | >90% |
| | | GsGsUCUCAfCfUfUUCCAGCAAA- | | | |
| | | C6-NH-DBCO | | | |

### Example 11. Synthesis of AGT siRNA Duplex

Equimolar amounts of complementary sense and antisense strands were mixed and annealed by heating at 90° C. for 5 mins and slowly cooled to room temperature in 3 h to obtain the desired siRNAs (Table 1). Products were characterized and confirmed by HPLC (Agilent AdvanceBio Oligonucleotide, 4.6×150 mm, 0.6 mL/min, 100 mM TEAA pH7.0/100 mM TEAA pH7.0+90% ACN) and LC-MS.

TABLE 6
AGT siRNA duplexes (all riboses have 2′-OMe modifications except those with 2′ F,
see below table)

| SEQ ID | Orig. Seq. | | Mass. | Mass | HPLC |
|---|---|---|---|---|---|
| No. | Name | Sequence (5′-3′) | (Calc). | (Found) | purity |
| 127 | APG3M06 | SS: GalNAc-PDoV3- | 9964.81 + | 9967 + | >90% |
| | | GGGUCUCfAfCfUUUCCAG | 7095.12 | 7095 | |
| | | CsAsA | | | |
| 103 | | AS: | | | |
| | | PUsUfsGCUGGAAAGUGAf | | | |
| | | GACCsCsdTdT | | | |
| 128 | APG3M07 | SS: GalNAc-PDoV3- | 9948.81 + | 9950 + | >90% |
| | | GGUCUCAfCfUfUUCCAGC | 7096.11 | 7096 | |
| | | AsAsA | | | |
| 104 | | AS: | | | |
| | | PUsUfsUGCUGGAAAGUGf | | | |
| | | AGACsCsdTdT | | | |
| 73 | APG3M07a | SS: | 9873.77 | 9875 + | >90% |
| | | GsGsUCUCAfCfUfUUCCAG | 7096.11 | 7097 | |
| | | CAAA-PDoV3-GalNAc | | | |
| 105 | | AS: | | | |
| | | PUsUfsUGCUGGAAAGUGf | | | |
| | | AGACsCsdTdT | | | |
| 129 | APG3M14 | SS: GalNAc-PDoV3- | 9960.70 + | 9963 + | >90% |
| | | CCAGUUUfGfCfUGGGUU | 7069.18 | 7069 | |
| | | UAsUsU | | | |
| 106 | | AS: | | | |
| | | PAsAfsUAAACCCAGCAAf | | | |
| | | ACUGsGsdTdT | | | |
| 130 | APG3M21 | SS: GalNAc-PDoV3- | 10686.29 + | 10688 + | >90% |
| | | GUCAUCCfACfAfAfUGAG | 7755.48 | 7755 | |
| | | AGUAsCsA | | | |
| 107 | | AS: | | | |
| | | PUsGfsUACUCUCAUUGUf | | | |
| | | GGfAUGACsGsA | | | |
| 131 | APG3M21a | SS: GalNAc-PDoV3- | 10698.21 + | 10700 | >90% |
| | | GUCAUCCfAfCfAAUGAGA | 7767.50 | 7767 | |
| | | GUASCSA | | | |
| 108 | | AS: | | | |
| | | PUsGfsUACUCUCAUUGUf | | | |
| | | GGAUGACsGsA | | | |

All riboses are 2′-OMe, except f is 2′-F modification. S is PS modification. P is phospholation. Table 2A shows SEQ ID Nos 127, 128, 129, 130 and 131, as SEQ ID Nos. 71, 72, 74, 75, and 76, respectively, with all modifications in the sequences themselves.

1. A conjugate comprising a peptide covalently coupled to an siRNA duplex and an N-acetyl-galactosamine (GalNAc) moiety, wherein said siRNA duplex consists essentially of a duplex comprising 14-25 duplex residues of an siRNA selected from the group consisting of (a) AGT01-AGT22 (SEQ ID Nos. 1, 23; 2, 24; 3, 25; 4, 26; 5, 27; 6, 28; 7, 29; 8, 30; 9, 31; 10, 32; 11, 33; 12, 34; 13, 35; 14, 36; 15, 37; 16, 38; 17, 39; 18, 40; 19, 41; 20, 42; 21, 43; and 22, 44, respectively), wherein said duplex is modified or unmodified and (b) M02 (SEQ ID Nos. 45, 77), M04 (SEQ ID Nos. 46, 78); M05 (SEQ ID Nos. 47, 79); M06 (SEQ ID Nos. 48, 80); M07 (SEQ ID Nos. 49, 81); M07a (SEQ ID Nos. 113, 114); M07c1 (SEQ ID Nos. 50, 82); M07d1 (SEQ ID Nos. 51, 83); M07f1 (SEQ ID Nos. 52, 84); M07m (SEQ ID Nos. 53, 85); M07m1 (SEQ ID Nos. 54, 86); M07m2 (SEQ ID Nos. 55, 87); M08 (SEQ ID Nos. 57, 88); M09 (SEQ ID Nos. 57, 89); M10 (SEQ ID Nos. 58, 90); M11 (SEQ ID Nos. 59, 91); M12 (SEQ ID Nos. 60, 92); M13 (SEQ ID Nos. 61, 93); M14 (SEQ ID Nos. 62, 94); M15 (SEQ ID Nos. 63, 95); M16 (SEQ ID Nos. 64, 96), and APG3M06 (SEQ ID Nos. 71, 103); APG3M07 (SEQ ID Nos. 72, 104); APG3M07a, (SEQ ID No. 73, 105); and APG3M14 (SEQ ID Nos 74, 106).

2. A conjugate according to claim 1 wherein said siRNA duplex consists essentially of a duplex comprising 14-25 duplex residues of an siRNA selected from the group consisting of AGT01-AGT20 (SEQ ID Nos. 1-44), wherein at least one residue of said duplex is modified.

3. The conjugate of claim 2, wherein said modification is a 2′-O-methyl or 2′-F modification.

4. A conjugate according to claim 2 wherein said siRNA duplex is selected from the group consisting of M06 (SEQ ID Nos. 48, 80), M07 (SEQ ID Nos. 49, 81) and M07a (SEQ ID Nos. 113, 114).

5. A conjugate according to claim 2 wherein said siRNA duplex is M14 (SEQ ID Nos. 62, 94), wherein the modified oligonucleotide comprises at least one modification selected from a modified GalNAc moiety and a peptide linkage.

6. The conjugate according to claim 1, wherein said siRNA comprises a sense strand that is at least 90% complementary to a nucleobase antisense sequence selected from the group consisting of the antisense sequences of AGT01-AGT22 (SEQ ID Nos. 23-44).

7. The conjugate according to claim 1, comprising two or more or three or more GalNAc moieties.

8. The conjugate according to claim 1, wherein said siRNA comprises a dTdT overhang at the 3′ end of one or both of the strands, a phosphate modification at the 5′ end of the antisense strandand/or at least two phosphorothioates modifications at the end of at least one strand.

9. The conjugate according to claim 1, wherein the siRNA molecule is coupled via the 5′ end of the sense strand or via the 3′ end of one strand.

10. The conjugate according to claim 1, wherein the modified oligonucleotide comprises a trivalent GalNAc motif having the structure:

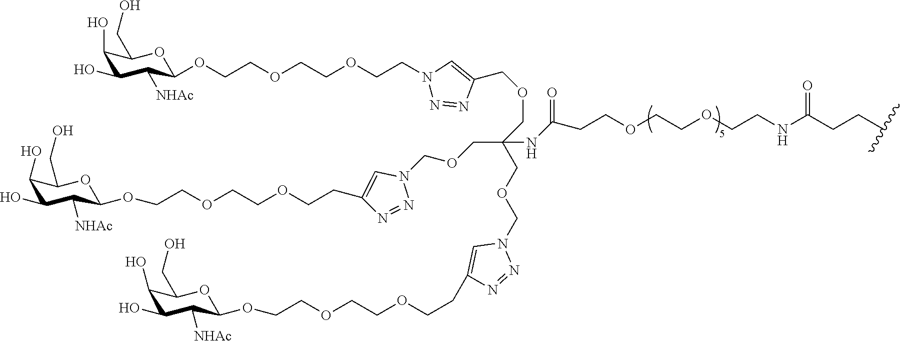

11. The conjugate according to claim 1, comprising a peptide docking vehicle having the structure disclosed as SEQ ID NO: 135:

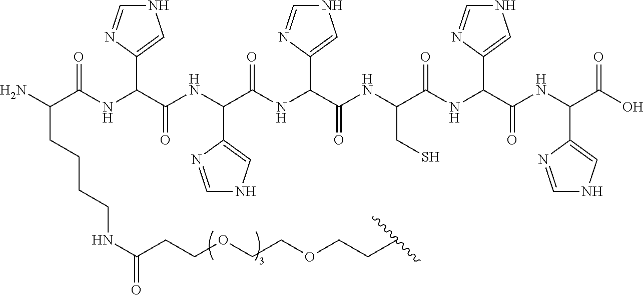

12. The conjugate according to claim 1, wherein the peptide is coupled via a peg linker covalently linked to the side chain of a lysine residue.

13. The conjugate of claim 12, wherein the peg linker comprises —(CH₂CH₂)₃—OCH₂CH₂—N—.

14. A conjugate according to claim 1, wherein the peptide comprises a cysteine residue.

15. A conjugate according claim 1 having the structure disclosed as SEQ ID NO: 136:

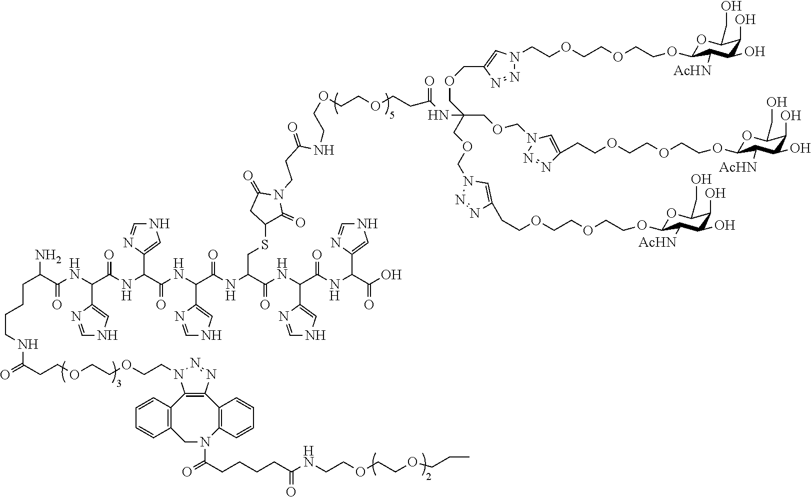

16. A pharmaceutical composition comprising the conjugate according to claim 1 and a pharmaceutically acceptable carrier or diluent.

17. The composition according to claim 16, further comprising an HKP copolymer.

18. The composition according to claim 17 wherein said conjugate and said copolymer are formulated into nanoparticles.

19. A method of treating hypertension in a subject comprising administering to said subject an effective amount of a conjugate according to claim 1 or a pharmaceutical composition comprising said conjugate and a pharmaceutically acceptable carrier or diluent.

20. The method of claim 19, wherein said subject is a non-human primate or a human.
